(19) United States
(12) Patent Application Publication
(10) Pub. No.: US 2025/0041321 A1
(43) Pub. Date: Feb. 6, 2025

(54) SYSTEMS FOR TREATING PULMONARY INFECTIONS

(71) Applicant: Insmed Incorporated, Bridgewater, NJ (US)

(72) Inventors: Walter PERKINS, Bridgewater, NJ (US); Vladimir MALININ, Bridgewater, NJ (US); Xingong LI, Bridgewater, NJ (US); Brian MILLER, Bridgewater, NJ (US); Dominique SEIDEL, Bridgewater, NJ (US); Philipp HOLZMANN, Bridgewater, NJ (US); Harald SCHULZ, Bridgewater, NJ (US); Michael HAHN, Bridgewater, NJ (US)

(73) Assignee: Insmed Incorporated, Bridgewater, NJ (US)

(21) Appl. No.: 18/733,525

(22) Filed: Jun. 4, 2024

Related U.S. Application Data
(63) Continuation of application No. 18/142,532, filed on May 2, 2023, now abandoned.
Application No. 18/142,532 is a continuation of application No. 17/175,317, filed on Feb. 12, 2021, now abandoned.
Application No. 17/175,317 is a continuation of application No. 16/919,771, filed on Jul. 2, 2020, now abandoned.
Application No. 16/919,771 is a continuation of application No. 16/682,674, filed on Nov. 13, 2019, now abandoned.
Application No. 16/682,674 is a continuation of application No. 16/010,094, filed on Jun. 15, 2018, now abandoned.
Application No. 16/010,094 is a continuation of application No. 15/900,261, filed on Feb. 20, 2018, now abandoned.
Application No. 15/900,261 is a continuation of application No. 15/379,804, filed on Dec. 15, 2016, now abandoned.
Application No. 15/379,804 is a continuation of application No. 13/899,457, filed on May 21, 2013, now Pat. No. 9,566,234.
(60) Provisional application No. 61/649,830, filed on May 21, 2012.

(51) Int. Cl. A61K 31/7036; A61K 9/00; A61K 9/127; A61M 11/00; A61M 11/02

(52) U.S. Cl. CPC A61K 31/7036; A61K 9/0078; A61K 9/127; A61K 9/1271; A61M 11/005; A61M 11/02

(57) ABSTRACT

Provided herein are systems for treating a subject with a pulmonary infection, for example, a nontuberculous mycobacterial pulmonary infection, a Burkholderia pulmonary infection, a pulmonary infection associated with bronchiectasis, or a Pseudomonas pulmonary infection. The system includes a pharmaceutical formulation comprising a liposomal aminoglycoside dispersion, and the lipid component of the liposomes consist essentially of electrically neutral lipids. The system also includes a nebulizer which generates an aerosol of the pharmaceutical formulation at a rate greater than about 0.53 gram per minute. The aerosol is delivered to the subject via inhalation for the treatment of the pulmonary infection.

### CROSS REFERENCE TO RELATED APPLICATIONS

This application is a continuation of U.S. application Ser. No. 18/142,532, filed May 2, 2023, which is a continuation of U.S. application Ser. No. 17/175,317, filed Feb. 12, 2021, now abandoned, which is a continuation of U.S. application Ser. No. 16/919,771, filed Jul. 2, 2020, now abandoned, which is a continuation of U.S. application Ser. No. 16/682,674, filed Nov. 13, 2019, now abandoned, which is a continuation of U.S. application Ser. No. 16/010,094, filed Jun. 15, 2018, now abandoned, which is a continuation of U.S. application Ser. No. 15/900,261, filed Feb. 20, 2018, now abandoned, which is a continuation of U.S. application Ser. No. 15/379,804, filed Dec. 15, 2016, now abandoned, which is a continuation of U.S. application Ser. No. 13/899,457, filed May 21, 2013, now U.S. Pat. No. 9,566,234, which claims the benefit of priority from U.S. Provisional Application No. 61/649,830, filed May 21, 2012, each of which is hereby incorporated by reference in its entirety.

### BACKGROUND OF THE INVENTION

Certain technologies suitable for administration by inhalation employ liposomes and lipid complexes supply a prolonged therapeutic effect of drug in the lung. These technologies also provide the drug with sustained activities, and the ability to target and enhance the uptake of the drug into sites of disease.

Inhalation delivery of liposomes is complicated by their sensitivity to shear-induced stress during nebulization, which can lead to change in physical characteristics (e.g., entrapment, size). However, as long as the changes in characteristics are reproducible and meet acceptability criteria, they need not be prohibitive to pharmaceutical development.

Cystic fibrosis (CF) patients have thick mucus and/or sputum secretions in the lungs, frequent consequential infections, and biofilms resulting from bacterial colonizations. All these fluids and materials create barriers to effectively targeting infections with aminoglycosides. Liposomal aminoglycoside formulations may be useful in combating the bacterial biofilms.

### SUMMARY OF THE INVENTION

The present invention provides methods for treating various pulmonary infections, including mycobacterial infections (e.g., pulmonary infections caused by nontuberculous Mycobacterium, also referred to herein as nontuberculous mycobacterial (NTM) infections), by providing systems for delivery of aerosolized liposomal formulations via inhalation. For example, the systems and methods provided herein can be used to treat a pulmonary nontuberculous mycobacterial infection such as pulmonary M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilusenavense, M. gordonae, M. ulcerans, M. fortuitum or M. fortuitum complex (M. fortuitum and M. chelonae) infection.

In one aspect, the present invention provides a system for treating or providing prophylaxis against a pulmonary infection. In one embodiment, the system comprises a pharmaceutical formulation comprising a liposomal complexed aminoglycoside, wherein the formulation is a dispersion (e.g., a liposomal solution or suspension), the lipid component of the liposome consists of electrically neutral lipids, and a nebulizer which generates an aerosol of the pharmaceutical formulation at a rate greater than about 0.53 g per minute. In one embodiment, the mass median acrodynamic diameter (MMAD) of the aerosol is less than about 4.2 μm, as measured by the Anderson Cascade Impactor (ACI), about 3.2 μm to about 4.2 μm, as measured by the ACI, or less than about 4.9 μm, as measured by the Next Generation Impactor (NGI), or about 4.4 μm to about 4.9 μm, as measured by the NGI.

In another embodiment, the system for treating or providing prophylaxis against a pulmonary infection comprises a pharmaceutical formulation comprising a liposomal complexed aminoglycoside, wherein the formulation is a dispersion (e.g., a liposomal solution or suspension), the lipid component of the liposome consists of electrically neutral lipids, and a nebulizer which generates an acrosol of the pharmaceutical formulation at a rate greater than about 0.53 g per minute. The fine particle fraction (FPF) of the aerosol is greater than or equal to about 64%, as measured by the Anderson Cascade Impactor (ACI), or greater than or equal to about 51%, as measured by the Next Generation Impactor (NGI).

In one embodiment, the system provided herein comprises a pharmaceutical formulation comprising an aminoglycoside. In a further embodiment, the aminoglycoside is amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin or a combination thereof. In even a further embodiment, the aminoglycoside is amikacin. In another embodiment, the aminoglycoside is selected from an aminoglycoside set forth in Table A, below, or a combination thereof.

TABLE A

AC4437
amikacin
apramycin
arbekacin
astromicin
bekanamycin
boholmycin
brulamycin
capreomycin
dibekacin
dactimicin
etimicin
framycetin
gentamicin
H107
hygromycin
hygromycin B
inosamycin
K-4619
isepamicin
KA-5685
kanamycin
neomycin
netilmicin
paromomycin
plazomicin
ribostamycin
sisomicin
rhodestreptomycin
sorbistin
spectinomycin
sporaricin
streptomycin
tobramycin
verdamicin
vertilmicin

The pharmaceutical formulations provided herein are dispersions of liposomes (i.e., liposomal dispersions or aqueous liposomal dispersions which can be either liposomal solutions or liposomal suspensions). In one embodiment, the lipid component of the liposomes consists essentially of one or more electrically neutral lipids. In a further embodiment, the electrically neutral lipid comprises a phospholipid and a sterol. In a further embodiment, the phospholipid is dipalmitoylphosphatidylcholine (DPPC) and the sterol is cholesterol.

In one embodiment, the lipid to drug ratio in the aminoglycoside pharmaceutical formulation (aminoglycoside liposomal solution or suspension) is about 2:1, about 2:1 or less, about 1:1, about 1:1 or less, or about 0.7:1.

In one embodiment, the aerosolized aminoglycoside formulation, upon nebulization, has an aerosol droplet size of about 1 μm to about 3.8 μm, about 1.0 μm to 4.8 μm, about 3.8 μm to about 4.8 μm, or about 4.0 μm to about 4.5 μm. In a further embodiment, the aminoglycoside is amikacin. In even a further embodiment, the amikacin is amikacin sulfate.

In one embodiment, about 70% to about 100% of the aminoglycoside present in the formulation is liposomal complexed, e.g., encapsulated in a plurality of liposomes, prior to nebulization. In a further embodiment, the aminoglycoside is selected from an aminoglycoside provided in Table A. In further embodiment, the aminoglycoside is an amikacin. In even a further embodiment, about 80% to about 100% of the amikacin is liposomal complexed, or about 80% to about 100% of the amikacin is encapsulated in a plurality of liposomes. In another embodiment, prior to nebulization, about 80% to about 100%, about 80% to about 99%, about 90% to about 100%, 90% to about 99%, or about 95% to about 99% of the aminoglycoside present in the formulation is liposomal complexed prior to nebulization.

In one embodiment, the percent liposomal complexed (also referred to herein as “liposomal associated”) aminoglycoside post-nebulization is from about 50% to about 80%, from about 50% to about 75%, from about 50% to about 70%, from about 55% to about 75%, or from about 60% to about 70%. In a further embodiment, the aminoglycoside is selected from an aminoglycoside provided in Table A. In a further embodiment, the aminoglycoside is amikacin. In even a further embodiment, the amikacin is amikacin sulfate.

In another aspect, the present invention provides methods for treating or providing prophylaxis against a pulmonary infection. In one embodiment, the pulmonary infection is a pulmonary infection caused by a gram negative bacterium (also referred to herein as a gram negative bacterial infection). In one embodiment, the pulmonary infection is a Pseudomonas infection, e.g., a Pseudomonas aeruginosa infection. In another embodiment, the pulmonary infection is caused by one of the Pseudomonas species provided in Table B, below. In one embodiment, a patient is treated for mycobacterial lung infection with one of the systems provided herein. In a further embodiment, the mycobacterial pulmonary infection is a nontuberculous mycobacterial pulmonary infection, a Mycobacterium abscessus pulmonary infection or a Mycobacterium avium complex pulmonary infection. In one or more of the preceding embodiments, the patient is a cystic fibrosis patient.

In one embodiment, a patient with cystic fibrosis is treated for a pulmonary infection with one of the systems provided herein. In a further embodiment, the pulmonary infection is caused by Mycobacterium abscessus, Mycobacterium avium complex, or P. aeruginosa. In another embodiment, the pulmonary infection is caused by a nontuberculous Mycobacterium selected from M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilus. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae) or a combination thereof.

In another aspect, a method for treating or providing prophylaxis against a pulmonary infection in a patient is provided. In one embodiment, the method comprises aerosolizing a pharmaceutical formulation comprising a liposomal complexed aminoglycoside, wherein the pharmaceutical formulation is an aqueous dispersion of liposomes (e.g., a liposomal solution or liposomal suspension), and is aerosolized at a rate greater than about 0.53 gram per minute. The method further comprises administering the aerosolized pharmaceutical formulation to the lungs of the patient; wherein the aerosolized pharmaceutical formulation comprises a mixture of free aminoglycoside and liposomal complexed aminoglycoside, and the lipid component of the liposome consists of electrically neutral lipids. In a further embodiment, the mass median acrodynamic diameter (MMAD) of the acrosol is about 1.0 μm to about 4.2 μm as measured by the ACI. In any one of the proceeding embodiments, the MMAD of the aerosol is about 3.2 μm to about 4.2 μm as measured by the ACI. In any one of the proceeding embodiments, the MMAD of the aerosol is about 1.0 μm to about 4.9 μm as measured by the NGI. In any one of the proceeding embodiments, the MMAD of the acrosol is about 4.4 μm to about 4.9 μm as measured by the NGI.

In one embodiment, the method comprises aerosolizing a pharmaceutical formulation comprising a liposomal complexed aminoglycoside, wherein the pharmaceutical formulation is an aqueous dispersion and is aerosolized at a rate greater than about 0.53 gram per minute. The method further comprises administering the aerosolized pharmaceutical formulation to the lungs of the patient; wherein the aerosolized pharmaceutical formulation comprises a mixture of free aminoglycoside and liposomal complexed aminoglycoside (e.g., aminoglycoside encapsulated in a liposome), and the liposome component of the formulation consists of electrically neutral lipids. In even a further embodiment, fine particle fraction (FPF) of the aerosol is greater than or equal to about 64%, as measured by the ACI, or greater than or equal to about 51%, as measured by the NGI.

In another aspect, a liposomal complexed aminoglycoside aerosol (e.g., a liposomal complexed aminoglycoside) is provided. In one embodiment, the acrosol comprises an aminoglycoside and a plurality of liposomes comprising DPPC and cholesterol, wherein about 65% to about 75% of the aminoglycoside is liposomal complexed and the acrosol is generated at a rate greater than about 0.53 gram per minute. In a further embodiment, about 65% to about 75% of the aminoglycoside is liposomal complexed, and the aerosol is generated at a rate greater than about 0.53 gram per minute. In any one of the proceeding embodiments, the aerosol is generated at a rate greater than about 0.54 gram per minute. In any one of the proceeding embodiments, the acrosol is generated at a rate greater than about 0.55 gram per minute. In any one of the preceding embodiments, the aminoglycoside is selected from an aminoglycoside provided in Table A.

In one embodiment, the MMAD of the liposomal complexed aminoglycoside aerosol is about 3.2 μm to about 4.2 μm, as measured by the ACI, or about 4.4 μm to about 4.9 μm, as measured by the NGI. In a further embodiment, the aerosol comprises an aminoglycoside and a plurality of liposomes comprising DPPC and cholesterol, wherein about 65% to about 75% of the aminoglycoside is liposomal complexed (e.g., encapsulated in the plurality of the liposomes), and the liposomal aminoglycoside acrosol is generated at a rate greater than about 0.53 gram per minute. In a further embodiment, the aminoglycoside is selected from an aminoglycoside provided in Table A.

In one embodiment, the FPF of the lipid-complexed aminoglycoside aerosol is greater than or equal to about 64%, as measured by the Anderson Cascade Impactor (ACI), or greater than or equal to about 51%, as measured by the Next Generation Impactor (NGI). In a further embodiment, the aerosol comprises an aminoglycoside and a plurality of liposomes comprising DPPC and cholesterol, wherein about 65% to about 75% of the aminoglycoside is liposomal complexed, for example, encapsulated in the plurality of the liposomes, and the liposomal aminoglycoside aerosol is generated at a rate greater than about 0.53 gram per minute. In any one of the proceeding embodiments, the aerosol is generated at a rate greater than about 0.54 gram per minute. In any one of the proceeding embodiments, the aerosol is generated at a rate or greater than about 0.55 gram per minute. In any of the preceding embodiments, the aminoglycoside is selected from an aminoglycoside provided in Table A.

In one embodiment, the aerosol comprises an aminoglycoside and a plurality of liposomes comprising DPPC and cholesterol, wherein about 65% to about 75% of the aminoglycoside is liposomal complexed. In a further embodiment, about 65% to about 75% of the aminoglycoside is encapsulated in the plurality of liposomes. In a further embodiment, the aerosol is generated at a rate greater than about 0.53 gram per minute, greater than about 0.54 gram per minute, or greater than about 0.55 gram per minute. In a further embodiment, the aminoglycoside is amikacin (e.g., amikacin sulfate).

In one embodiment, the concentration of the aminoglycoside in the liposomal complexed aminoglycoside is about 50 mg/mL or greater. In a further embodiment, the concentration of the aminoglycoside in the liposomal complexed aminoglycoside is about 60 mg/mL or greater. In a further embodiment, the concentration of the aminoglycoside in the liposomal complexed aminoglycoside is about 70 mg/mL or greater, for example about 70 mg/mL to about 75 mg/mL. In a further embodiment, the aminoglycoside is selected from an aminoglycoside provided in Table A. In even a further embodiment, the aminoglycoside is amikacin (e.g., amikacin sulfate).

### BRIEF DESCRIPTION OF THE FIGURES

FIG. 1 shows a diagram of a nebulizer (aerosol generator) in which the present invention may be implemented.

FIG. 2 is an enlarged representation of the nebulizer diagram shown in FIG. 1.

FIG. 3 shows a cross-sectional view of a generally known aerosol generator, as described in WO 2001/032246.

FIG. 4 is an image of a PARI eFlow® nebulizer, modified for use with the aminoglycoside formulations described herein, and a blown up diagram of the nebulizer's membrane.

FIG. 5 is a cross-sectional computed tomography (CT) image showing a membrane having a relatively long nozzle portion.

FIG. 6 is a cross-sectional computed tomography (CT) image of a stainless steel membrane having a relatively short nozzle portion.

FIG. 7 is a cross sectional cartoon depiction of the sputum/biofilm seen, for example, in patients with cystic fibrosis.

FIG. 8 is a graph of the time period of aerosol generation upon complete emission of the liquid within the liquid reservoir (Nebulization time) as a function of the initial gas cushion within the liquid reservoir (V_{A}).

FIG. 9 is a graph of negative pressure in the nebulizer as a function of the time of aerosol generation until complete emission of the pharmaceutical formulation from the liquid reservoir (nebulization time).

FIG. 10 is a graph of aerosol generation efficiency as a function of the negative pressure in the nebulizer.

FIG. 11 is a graph of the period of time for aerosol generation upon complete emission of the liquid (nebulization time) as a function of the ratio between the increased volume V_{RN} of the liquid reservoir and the initial volume of liquid within the liquid reservoir (V_{L})(V_{RN}/V_{L}).

FIG. 12 is a graph showing the MMAD of aerosolized formulations as a function of nebulization rate of the respective formulation.

FIG. 13 is a graph showing the FPF of aerosolized formulations as a function of the nebulization rate of the respective formulation.

FIG. 14 is a schematic of the system used for the recovery of aerosol for post-nebulization studies.

### DETAILED DESCRIPTION OF THE INVENTION

The invention described herein is directed, in part, to systems for administering an aminoglycoside pharmaceutical formulation to the lungs of a subject, for example, to treat a pulmonary disorder.

The term “treating” includes: (1) preventing or delaying the appearance of clinical symptoms of the state, disorder or condition developing in the subject that may be afflicted with or predisposed to the state, disorder or condition but does not yet experience or display clinical or subclinical symptoms of the state, disorder or condition; (2) inhibiting the state, disorder or condition (i.e., arresting, reducing or delaying the development of the disease, or a relapse thereof in case of maintenance treatment, of at least one clinical or subclinical symptom thereof); and/or (3) relieving the condition (i.e., causing regression of the state, disorder or condition or at least one of its clinical or subclinical symptoms). The benefit to a subject to be treated is either statistically significant or at least perceptible to the subject or to the physician.

In one embodiment, pulmonary infections caused by the following bacteria are treatable with the systems and formulations provided herein: Pseudomonas (e.g., P. aeruginosa, P. paucimobilis, P. putida, P. fluorescens, and P. acidovorans), Burkholderia (e.g., B. pseudomallei, B. cepacia, B. cepacia complex, B. dolosa, B. fungorum, B. gladioli, B. multivorans, B. vietnamiensis, B. pseudomallei, B. ambifaria, B. andropogonis, B. anthina, B. brasilensis, B. caledonica, B. caribensis, B. caryophylli), Staphylococcus (e.g., S. aureus, S. auricularis, S. carnosus, S. epidermidis, S. lugdunensis), Methicillin-resistant Staphylococcus aureus (MRSA), Streptococcus (e.g., Streptococcus pneumoniae), Escherichia coli, Klebsiella, Enterobacter, Serratia, Haemophilus, Yersinia pestis, Mycobacterium (e.g., nontuberculous Mycobacterium).

In one embodiment, a patient is treated for a nontuberculous mycobacterial lung infection with one of the systems provided herein. In a further embodiment, the nontuberculous mycobacterial lung infection is a recalcitrant nontuberculous mycobacterial lung infection.

In one embodiment, the systems provided herein are used to treat a patient having a pulmonary infection caused by Pseudomonas. In a further embodiment, the pulmonary infection is caused by a Pseudomonas species selected from a species provided in Table B, below.

TABLE B

| | | | | | |
|---|---|---|---|---|---|
| P. abietaniphila | P. aeruginosa | P. agarici | P. alcaligenes | P. alcaliphila | P. amygdale |
| P. anguilliseptica | P. antarctica | P. argentinensis | P. asplenii | P. aurantiaca | P. aureofaciens |
| P. avellanae | P. azotifigens | P. azotoformans | P. balearica | P. borbori | P. brassicacearum |
| P. brenneri | P. cannabina | P. caricapapayae | P. cedrina | P. chlororaphis | P. cichorii |
| P. citronellolis | P. coenobios | P. congelans | P. coronofaciens | P. corrugate | P. costantinii |
| P. cremoricolorata | P. cruciviae | P. delhiensis | P. denitrificans | P. excibis | P. extremorientalis |
| P. ficuseructae | P. flavescens | P. fluorescens | P. fragi | P. frederiksbergensis | P. fulva |
| P. fuscovaginae | P. gelidicola | P. gessardii | P. grimontii | P. indica | P. jessenii |
| P. jinjuensis | P. kilonensis | P. knackmussii | P. koreensis | P. libanensis | P. lini |
| P. lundensis | P. lutea | P. luteola | P. mandelii | P. marginalis | P. mediterranea |
| P. meliae | P. mendocina | P. meridiana | P. migulae | P. monteilii | P. moraviensis |
| P. mosselii | P. mucidolens | P. nitroreducens | P. oleovorans | P. orientalis | P. oryzihabitans |
| P. otitidis | P. pachastrellae | P. palleroniana | P. panacis | P. papaveris | P. parafulva |
| P. peli | P. perolens | P. pertucinogena | P. plecoglossicida | P. poae | P. pohangensis |
| P. proteolytica | P. pseudoalcaligenes | P. psychrophila | P. psychrotolerans | P. putida | P. rathonis |
| P. reptilivora | P. resiniphila | P. resinovorans | P. rhizosphaerae | P. rhodesiae | P. rubescens |
| P. salomonii | P. savastanoi | P. segitis | P. septic | P. simiae | P. straminea |
| P. stutzeri | P. suis | P. synxantha | P. syringae | P. taetrolens | P. thermotolerans |
| P. thivervalensis | P. tolaasii | P. tremae | P. trivialis | P. turbinellae | P. tuticorinensis |
| P. umsongensis | P. vancouverensis | P. veronii | P. viridiflava | P. vranovensis | P. xanthomarina |

The nontuberculous mycobacterial lung infection, in one embodiment, is selected from M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilus. genavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae) or a combination thereof. In a further embodiment, the nontuberculous mycobacterial lung infection is M. abscessus or M. avium. In a further embodiment, the M. avium infection is M. avium subsp. hominissuis. In one embodiment, the nontuberculous mycobacterial lung infection is a recalcitrant nontuberculous mycobacterial lung infection.

In another embodiment, a cystic fibrosis patient is treated for a bacterial infection with one of the systems provided herein. In a further embodiment, the bacterial infection is a lung infection due to Pseudomonas aeruginosa. In yet another embodiment, a patient is treated for a pulmonary infection associated with bronchiectasis with one of the systems provided herein.

“Prophylaxis,” as used herein, can mean complete prevention of an infection or disease, or prevention of the development of symptoms of that infection or disease; a delay in the onset of an infection or disease or its symptoms; or a decrease in the severity of a subsequently developed infection or disease or its symptoms.

The term “antibacterial” is art-recognized and refers to the ability of the compounds of the present invention to prevent, inhibit or destroy the growth of microbes of bacteria. Examples of bacteria are provided above.

The term “antimicrobial” is art-recognized and refers to the ability of the aminoglycoside compounds of the present invention to prevent, inhibit, delay or destroy the growth of microbes such as bacteria, fungi, protozoa and viruses.

“Effective amount” means an amount of an aminoglycoside (e.g., amikacin) used in the present invention sufficient to result in the desired therapeutic response. The effective amount of the formulation provided herein comprises both free and liposomal complexed aminoglycoside. For example, the liposomal complexed aminoglycoside, in one embodiment, comprises aminoglycoside encapsulated in a liposome, or complexed with a liposome, or a combination thereof.

In one embodiment, the aminoglycoside is selected from amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin or verdamicin. In another embodiment, the aminoglycoside is selected from an aminoglycoside set forth in Table C, below.

TABLE C

AC4437
amikacin
arbekacin
apramycin
astromicin
bekanamycin
boholmycin
brulamycin
capreomycin
dibekacin
dactimicin
etimicin
framycetin
gentamicin
H107
hygromycin
hygromycin B
inosamycin
K-4619
isepamicin
KA-5685
kanamycin
neomycin
netilmicin
paromomycin
plazomicin
ribostamycin
sisomicin
rhodestreptomycin
sorbistin
spectinomycin
sporaricin
streptomycin
tobramycin
verdamicin
vertilmicin

In one embodiment, the aminoglycoside is an aminoglycoside free base, or its salt, solvate, or other non-covalent derivative. In a further embodiment, the aminoglycoside is amikacin. Included as suitable aminoglycosides used in the drug formulations of the present invention are pharmaceutically acceptable addition salts and complexes of drugs. In cases where the compounds may have one or more chiral centers, unless specified, the present invention comprises each unique racemic compound, as well as each unique nonracemic compound. In cases in which the active agents have unsaturated carbon-carbon double bonds, both the cis (Z) and trans (E) isomers are within the scope of this invention. In cases where the active agents exist in tautomeric forms, such as keto-enol tautomers, each tautomeric form is contemplated as being included within the invention. Amikacin, in one embodiment, is present in the pharmaceutical formulation as amikacin base, or amikacin salt, for example, amikacin sulfate or amikacin disulfate. In one embodiment, a combination of one or more of the above aminoglycosides is used in the formulations, systems and methods described herein. In a further embodiment, the combination comprises amikacin.

The therapeutic response can be any response that a user (e.g., a clinician) will recognize as an effective response to the therapy. The therapeutic response will generally be a reduction, inhibition, delay or prevention in growth of or reproduction of one or more bacterium, or the killing of one or more bacterium, as described above. A therapeutic response may also be reflected in an improvement in pulmonary function, for example forced expiratory volume in one second (FEV₁). It is further within the skill of one of ordinary skill in the art to determine appropriate treatment duration, appropriate doses, and any potential combination treatments, based upon an evaluation of therapeutic response.

“Liposomal dispersion” refers to a solution or suspension comprising a plurality of liposomes.

An “aerosol”, as used herein, is a gaseous suspension of liquid particles. The aerosol provided herein comprises particles of the liposomal dispersion.

A “nebulizer” or an “aerosol generator” is a device that converts a liquid into an acrosol of a size that can be inhaled into the respiratory tract. Pneumonic, ultrasonic, electronic nebulizers, e.g., passive electronic mesh nebulizers, active electronic mesh nebulizers and vibrating mesh nebulizers are amenable for use with the invention if the particular nebulizer emits an aerosol with the required properties, and at the required output rate.

The process of pneumatically converting a bulk liquid into small droplets is called atomization. The operation of a pneumatic nebulizer requires a pressurized gas supply as the driving force for liquid atomization. Ultrasonic nebulizers use electricity introduced by a piezoelectric element in the liquid reservoir to convert a liquid into respirable droplets. Various types of nebulizers are described in Respiratory Care, Vol. 45, No. 6, pp. 609-622 (2000), the disclosure of which is incorporated herein by reference in its entirety. The terms “nebulizer” and “aerosol generator” are used interchangeably throughout the specification. “Inhalation device”, “inhalation system” and “atomizer” are also used in the literature interchangeably with the terms “nebulizer” and “aerosol generator”.

“Fine particle fraction” or “FPF”, as used herein, refers to the fraction of the aerosol having a particle size less than 5 μm in diameter, as measured by cascade impaction. FPF is usually expressed as a percentage.

“Mass median diameter” or “MMD” is determined by laser diffraction or impactor measurements, and is the average particle diameter by mass.

“Mass median aerodynamic diameter” or “MMAD” is normalized regarding the aerodynamic separation of aqua aerosol droplets and is determined impactor measurements, e.g., the Anderson Cascade Impactor (ACI) or the Next Generation Impactor (NGI). The gas flow rate, in one embodiment, is 28 Liter per minute by the Anderson Cascade Impactor (ACI) and 15 Liter per minute by the Next Generation Impactor (NGI). “Geometric standard deviation” or “GSD” is a measure of the spread of an acrodynamic particle size distribution.

In one embodiment, the present invention provides a system for treating a pulmonary infection or providing prophylaxis against a pulmonary infection. Treatment is achieved via delivery of the aminoglycoside formulation by inhalation via nebulization. In one embodiment, the pharmaceutical formulation comprises an aminoglycoside agent, e.g., an aminoglycoside.

The pharmaceutical formulation, as provided herein, is a liposomal dispersion. Specifically, the pharmaceutical formulation is a dispersion comprising a “liposomal complexed aminoglycoside” or an “aminoglycoside encapsulated in a liposome”. A “liposomal complexed aminoglycoside” includes embodiments where the aminoglycoside (or combination of aminoglycosides) is encapsulated in a liposome, and includes any form of aminoglycoside composition where at least about 1% by weight of the aminoglycoside is associated with the liposome either as part of a complex with a liposome, or as a liposome where the aminoglycoside may be in the aqueous phase or the hydrophobic bilayer phase or at the interfacial headgroup region of the liposomal bilayer.

In one embodiment, the lipid component of the liposome comprises electrically neutral lipids, positively charged lipids, negatively charged lipids, or a combination thereof. In another embodiment, the lipid component comprises electrically neutral lipids. In a further embodiment, the lipid component consists essentially of electrically neutral lipids. In even a further embodiment, the lipid component consists of electrically neutral lipids, e.g., a sterol and a phospholipid.

As provided above, liposomal complexed aminoglycoside embodiments include embodiments where the aminoglycoside is encapsulated in a liposome. In addition, the liposomal complexed aminoglycoside describes any composition, solution or suspension where at least about 1% by weight of the aminoglycoside is associated with the lipid either as part of a complex with the liposome, or as a liposome where the aminoglycoside may be in the aqueous phase or the hydrophobic bilayer phase or at the interfacial headgroup region of the liposomal bilayer. In one embodiment, prior to nebulization, at least about 5%, at least about 10%, at least about 20%, at least about 25%, at least about 50%, at least about 75%, at least about 80%, at least about 85%, at least about 90% or at least about 95% of the aminoglycoside in the formulation is so associated. Association, in one embodiment, is measured by separation through a filter where lipid and lipid-associated drug is retained (i.e., in the retentate) and free drug is in the filtrate.

The formulations, systems and methods provided herein comprise a lipid-encapsulated or lipid-associated aminoglycoside agent. The lipids used in the pharmaceutical formulations of the present invention can be synthetic, semi-synthetic or naturally-occurring lipids, including phospholipids, tocopherols, sterols, fatty acids, negatively-charged lipids and cationic lipids.

In one embodiment, at least one phospholipid is present in the pharmaceutical formulation. In one embodiment, the phospholipid is selected from: phosphatidylcholine (EPC), phosphatidylglycerol (PG), phosphatidylinositol (PI), phosphatidylserine (PS), phosphatidylethanolamine (PE), and phosphatidic acid (PA); the soya counterparts, soy phosphatidylcholine (SPC); SPG, SPS, SPI, SPE, and SPA; the hydrogenated egg and soya counterparts (e.g., HEPC, HSPC), phospholipids made up of ester linkages of fatty acids in the 2 and 3 of glycerol positions containing chains of 12 to 26 carbon atoms and different head groups in the 1 position of glycerol that include choline, glycerol, inositol, serine, ethanolamine, as well as the corresponding phosphatidic acids. The carbon chains on these fatty acids can be saturated or unsaturated, and the phospholipid may be made up of fatty acids of different chain lengths and different degrees of unsaturation.

In one embodiment, the pharmaceutical formulation includes dipalmitoylphosphatidylcholine (DPPC), a major constituent of naturally-occurring lung surfactant. In one embodiment, the lipid component of the pharmaceutical formulation comprises DPPC and cholesterol, or consists essentially of DPPC and cholesterol, or consists of DPPC and cholesterol. In a further embodiment, the DPPC and cholesterol have a mole ratio in the range of from about 19:1 to about 1:1, or about 9:1 to about 1:1, or about 4:1 to about 1:1, or about 2:1 to about 1:1, or about 1.86:1 to about 1:1. In even a further embodiment, the DPPC and cholesterol have a mole ratio of about 2:1 or about 1:1. In one embodiment, DPPC and cholesterol are provided in an aminoglycoside formulation, e.g., an aminoglycoside formulation.

Other examples of lipids for use with the invention include, but are not limited to, dimyristoylphosphatidycholine (DMPC), dimyristoylphosphatidylglycerol (DMPG), dipalmitoylphosphatidcholine (DPPC), dipalmitoylphosphatidylglycerol (DPPG), distearoylphosphatidylcholine (DSPC), distearoylphosphatidylglycerol (DSPG), dioleylphosphatidyl-ethanolamine (DOPE), mixed phospholipids such as palmitoylstearoylphosphatidyl-choline (PSPC), and single acylated phospholipids, for example, mono-oleoyl-phosphatidylethanolamine (MOPE).

In one embodiment, the at least one lipid component comprises a sterol. In a further embodiment, the at least one lipid component comprises a sterol and a phospholipid, or consists essentially of a sterol and a phospholipid, or consists of a sterol and a phospholipid. Sterols for use with the invention include, but are not limited to, cholesterol, esters of cholesterol including cholesterol hemi-succinate, salts of cholesterol including cholesterol hydrogen sulfate and cholesterol sulfate, ergosterol, esters of ergosterol including ergosterol hemi-succinate, salts of ergosterol including ergosterol hydrogen sulfate and ergosterol sulfate, lanosterol, esters of lanosterol including lanosterol hemi-succinate, salts of lanosterol including lanosterol hydrogen sulfate, lanosterol sulfate and tocopherols. The tocopherols can include tocopherols, esters of tocopherols including tocopherol hemi-succinates, salts of tocopherols including tocopherol hydrogen sulfates and tocopherol sulfates. The term “sterol compound” includes sterols, tocopherols and the like.

In one embodiment, at least one cationic lipid (positively charged lipid) is provided in the systems described herein. The cationic lipids used can include ammonium salts of fatty acids, phospholids and glycerides. The fatty acids include fatty acids of carbon chain lengths of 12 to 26 carbon atoms that are either saturated or unsaturated. Some specific examples include: myristylamine, palmitylamine, laurylamine and stearylamine, dilauroyl ethylphosphocholine (DLEP), dimyristoyl ethylphosphocholine (DMEP), dipalmitoyl ethylphosphocholine (DPEP) and distearoyl ethylphosphocholine (DSEP), N-(2,3-di-(9-(Z)-octadecenyloxy)-prop-1-yl-N,N,N-trimethylammoniu-m chloride (DOTMA) and 1,2-bis(oleoyloxy)-3-(trimethylammonio) propane (DOTAP).

In one embodiment, at least one anionic lipid (negatively charged lipid) is provided in the systems described herein. The negatively-charged lipids which can be used include phosphatidyl-glycerols (PGs), phosphatidic acids (PAs), phosphatidylinositols (PIs) and the phosphatidyl serines (PSs). Examples include DMPG, DPPG, DSPG, DMPA, DPPA, DSPA, DMPI, DPPI, DSPI, DMPS, DPPS and DSPS.

Without wishing to be bound by theory, phosphatidylcholines, such as DPPC, aid in the uptake of the aminoglycoside agent by the cells in the lung (e.g., the alveolar macrophages) and helps to maintain the aminoglycoside agent in the lung. The negatively charged lipids such as the PGs, PAs, PSs and PIs, in addition to reducing particle aggregation, are thought to play a role in the sustained activity characteristics of the inhalation formulation as well as in the transport of the formulation across the lung (transcytosis) for systemic uptake. The sterol compounds, without wishing to be bound by theory, are thought to affect the release characteristics of the formulation.

Liposomes are completely closed lipid bilayer membranes containing an entrapped aqueous volume. Liposomes may be unilamellar vesicles (possessing a single membrane bilayer) or multilamellar vesicles (onion-like structures characterized by multiple membrane bilayers, each separated from the next by an aqueous layer) or a combination thereof. The bilayer is composed of two lipid monolayers having a hydrophobic “tail” region and a hydrophilic “head” region. The structure of the membrane bilayer is such that the hydrophobic (nonpolar) “tails” of the lipid monolayers orient toward the center of the bilayer while the hydrophilic “heads” orient towards the aqueous phase.

Liposomes can be produced by a variety of methods (see, e.g., Cullis et al. (1987)). In one embodiment, one or more of the methods described in U.S. Patent Application Publication No. 2008/0089927 are used herein to produce the aminoglycoside encapsulated lipid formulations (liposomal dispersion). The disclosure of U.S. Patent Application Publication No. 2008/0089927 is incorporated by reference in its entirety for all purposes. For example, in one embodiment, at least one lipid and an aminoglycoside are mixed with a coacervate (i.e., a separate liquid phase) to form the liposome formulation. The coacervate can be formed to prior to mixing with the lipid, during mixing with the lipid or after mixing with the lipid. Additionally, the coacervate can be a coacervate of the active agent.

In one embodiment, the liposomal dispersion is formed by dissolving one or more lipids in an organic solvent forming a lipid solution, and the aminoglycoside coacervate forms from mixing an aqueous solution of the aminoglycoside with the lipid solution. In a further embodiment, the organic solvent is ethanol. In even a further embodiment, the one or more lipids comprise a phospholipid and a sterol.

In one embodiment, liposomes are produces by sonication, extrusion, homogenization, swelling, electroformation, inverted emulsion or a reverse evaporation method. Bangham's procedure (J. Mol. Biol. (1965)) produces ordinary multilamellar vesicles (MLVs). Lenk et al. (U.S. Pat. Nos. 4,522,803, 5,030,453 and 5,169,637), Fountain et al. (U.S. Pat. No. 4,588,578) and Cullis et al. (U.S. Pat. No. 4,975,282) disclose methods for producing multilamellar liposomes having substantially equal interlamellar solute distribution in each of their aqueous compartments. Paphadjopoulos et al., U.S. Pat. No. 4,235,871, discloses preparation of oligolamellar liposomes by reverse phase evaporation. Each of the methods is amenable for use with the present invention.

Unilamellar vesicles can be produced from MLVs by a number of techniques, for example, the extrusion techniques of U.S. Pat. No. 5,008,050 and U.S. Pat. No. 5,059,421. Sonication and homogenization cab be so used to produce smaller unilamellar liposomes from larger liposomes (sec, for example, Paphadjopoulos et al. (1968); Deamer and Uster (1983); and Chapman et al. (1968)).

The liposome preparation of Bangham et al. (J. Mol. Biol. 13, 1965, pp. 238-252) involves suspending phospholipids in an organic solvent which is then evaporated to dryness leaving a phospholipid film on the reaction vessel. Next, an appropriate amount of aqueous phase is added, the 60 mixture is allowed to “swell”, and the resulting liposomes which consist of multilamellar vesicles (MLVs) are dispersed by mechanical means. This preparation provides the basis for the development of the small sonicated unilamellar vesicles described by Papahadjopoulos et al. (Biochim. Biophys. Acta. 135, 1967, pp. 624-638), and large unilamellar vesicles.

Techniques for producing large unilamellar vesicles (LUVs), such as, reverse phase evaporation, infusion procedures, and detergent dilution, can be used to produce liposomes for use in the pharmaceutical formulations provided herein. A review of these and other methods for producing liposomes may be found in the text Liposomes, Marc Ostro, ed., Marcel Dekker, Inc., New York, 1983, Chapter 1, which is incorporated herein by reference. See also Szoka, Jr. et al., (Ann. Rev. Biophys. Bioeng. 9, 1980, p. 467), which is also incorporated herein by reference in its entirety for all purposes.

Other techniques for making liposomes include those that form reverse-phase evaporation vesicles (REV), U.S. Pat. No. 4,235,871. Another class of liposomes that may be used is characterized as having substantially equal lamellar solute distribution. This class of liposomes is denominated as stable plurilamellar vesicles (SPLV) as defined in U.S. Pat. No. 4,522,803, and includes monophasic vesicles as described in U.S. Pat. No. 4,588,578, and frozen and thawed multilamellar vesicles (FATMLV) as described above.

A variety of sterols and their water soluble derivatives such as cholesterol hemisuccinate have been used to form liposomes; see, e.g., U.S. Pat. No. 4,721,612. Mayhew et al., PCT Publication No. WO 85/00968, described a method for reducing the toxicity of drugs by encapsulating them in liposomes comprising alpha-tocopherol and certain derivatives thereof. Also, a variety of tocopherols and their water soluble derivatives have been used to form liposomes, see PCT Publication No. 87/02219.

The pharmaceutical formulation, in one embodiment, pre-nebulization, comprises liposomes with a mean diameter, that is measured by a light scattering method, of approximately 0.01 microns to approximately 3.0 microns, for example, in the range about 0.2 to about 1.0 microns. In one embodiment, the mean diameter of the liposomes in the formulation is about 200 nm to about 300 nm, about 210 nm to about 290 nm, about 220 nm to about 280 nm, about 230 nm to about 280 nm, about 240 nm to about 280 nm, about 250 nm to about 280 nm or about 260 nm to about 280 nm. The sustained activity profile of the liposomal product can be regulated by the nature of the lipid membrane and by inclusion of other excipients in the composition.

In order to minimize dose volume and reduce patient dosing time, in one embodiment, it is important that liposomal entrapment of the aminoglycoside (e.g., the aminoglycoside amikacin) be highly efficient and that the L/D ratio be at as low a value as possible and/or practical while keeping the liposomes small enough to penetrate patient mucus and biofilms, e.g., Pseudomonas biofilms. In one embodiment, the L/D ratio in liposomes provided herein is 0.7 or about 0.7 (w/w). In a further embodiment, the liposomes provided herein are small enough to effectively penetrate a bacterial biofilm (e.g., Pseudomonas biofilm). In even a further embodiment, the mean diameter of the liposomes, as measured by light scattering is about 260 to about 280 nm.

The lipid to drug ratio in the pharmaceutical formulations provided herein, in one embodiment, is 3 to 1 or less, 2.5 to 1 or less, 2 to 1 or less, 1.5 to 1 or less, or 1 to 1 or less. The lipid to drug ratio in the pharmaceutical formulations provided herein, in another embodiment, is less than 3 to 1, less than 2.5 to 1, less than 2 to 1, less than 1.5 to 1, or less than 1 to 1. In a further embodiment, the lipid to drug ratio is about 0.7 to or less or about 0.7 to 1. In one embodiment, one of the lipids or lipid combinations in Table 1, below, is used in the pharmaceutical formulation of the invention.

TABLE 1
Lipids amenable for use with the invention

| | Mole | Lipid/aminoglycoside |
|---|---|---|
| Lipid(s) | ratio | (w/w) |
| DPPC | — | 1.1 |
| DPPC/DOPG | 9:1 | 1.0 |
| DPPC/DOPG | 7:1 | 3.9 |
| DPPC/DOPG | 1:1 | 2.8 |
| DPPC/DOPG | 0.5:1 | 2.7 |
| DOPG | — | 2.6 |
| DPPC/Cholesterol | about 1:1 | about 0.7 |
| DPPC/Cholesterol | 1:1 | 0.7 |
| DPPC/Cholesterol | 19:1 | 1.0 |
| DPPC/Cholesterol | 9:1 | 1.2 |
| DPPC/Cholesterol | 4:1 | 1.7 |
| DPPC/Cholesterol | 1.86:1 | 2.1 |
| DPPC/Cholesterol | 1:1 | 2.7 |
| DPPC/DOPC/Cholesterol | 8.55:1:0.45 | 2.0 |
| DPPC/DOPC/Cholesterol | 6.65:1:0.35 | 3.0 |
| DPPC/DOPC/Cholesterol | 19:20:1 | 2.5 |
| DPPC/DOPC/Cholesterol | 8.55:1:0.45 | 3.8 |
| DPPC/DOPC/Cholesterol | 6.65:1:0.35 | 4.1 |
| DPPC/DOPC/Cholesterol | 19:20:1 | 4.2 |
| DPPC/DOPC/DOPG/Cholesterol | 42:4:9:45 | 3.7 |
| DPPC/DOPC/DOPG/Cholesterol | 59:5:6:30 | 3.7 |

In one embodiment, the system provided herein comprises an aminoglycoside formulation, for example, an amikacin formulation, e.g., amikacin base formulation. In one embodiment, the amount of aminoglycoside provided in the system is about 450 mg, about 500 mg, about 550 mg, about 560 mg, about 570 mg, about 580 mg, about 590 mg, about 600 mg or about 610 mg. In another embodiment, the amount of aminoglycoside provided in the system is from about 500 mg to about 600 mg, or from about 500 mg to about 650 mg, or from about 525 mg to about 625 mg, or from about 550 mg to about 600 mg. In one embodiment, the amount of aminoglycoside administered to the subject is about 560 mg and is provided in an 8 mL formulation. In one embodiment, the amount of aminoglycoside administered to the subject is about 590 mg and is provided in an 8 mL formulation. In one embodiment, the amount of aminoglycoside administered to the subject is about 600 mg and is provided in an 8 mL formulation. In one embodiment, the aminoglycoside is amikacin and the amount of amikacin provided in the system is about 450 mg, about 500 mg, about 550 mg, about 560 mg, about 570 mg, about 580 mg, about 590 mg, about 600 mg or about 610 mg. In another embodiment, the aminoglycoside is amikacin and the amount of amikacin provided in the system is from about 500 mg to about 650 mg, or from about 525 mg to about 625 mg, or from about 550 mg to about 600 mg. In one embodiment, the aminoglycoside is amikacin and the amount of amikacin administered to the subject is about 560 mg, and is provided in an 8 mL formulation. In one embodiment, the aminoglycoside is amikacin and the amount of amikacin administered to the subject is about 590 mg, and is provided in an 8 mL formulation. In one embodiment, the aminoglycoside is amikacin and the amount of aminoglycoside administered to the subject is about 600 mg and is provided in an 8 mL formulation.

In one embodiment, the system provided herein comprises an aminoglycoside formulation, for example, an amikacin (base formulation). In one embodiment, the aminoglycoside formulation provided herein comprises about 60 mg/mL aminoglycoside, about 65 mg/mL aminoglycoside, about 70 mg/mL aminoglycoside, about 75 mg/mL aminoglycoside, about 80 mg/mL aminoglycoside, about 85 mg/mL aminoglycoside, or about 90 mg/mL aminoglycoside. In a further embodiment, the aminoglycoside is amikacin.

In one embodiment, the system provided herein comprises an about 8 mL liposomal amikacin formulation. In one embodiment, the density of the liposomal amikacin formulation is about 1.05 gram/mL; and in one embodiment, approximately 8.4 grams of the liposomal amikacin formulation per dose is present in the system of the invention. In a further embodiment, the entire volume of the formulation is administered to a subject in need thereof.

In one embodiment, the pharmaceutical formulation provided herein comprises at least one aminoglycoside, at least one phospholipid and a sterol. In a further embodiment, the pharmaceutical formulation comprises an aminoglycoside, DPPC and cholesterol. In one embodiment, the pharmaceutical formulation is the formulation provided in Table 2, below.

TABLE 2
Pharmaceutical Formulations

| Component | Concentration | Component | Concentration | |
|---|---|---|---|---|
| Formulation A (pH 6.0-7.0) | | Formulation D (pH ~6.5) | | |
| Aminoglycoside | 60-80 mg/mL | Aminoglycoside | ~70 | mg/mL |
| Phospholipid | 30-40 mg/mL | Phospholipid | ~32-35 | mg/mL |
| Sterol | 10-20 mg/mL | Sterol | ~16-17 | mg/mL |
| Salt | 0.5%-5.0% | Salt | ~1.5% | |
| Formulation B (pH 6.0-7.0) | | Formulation E (pH ~6.5) | | |
| Amikacin | 60-80 mg/mL | Amikacin | ~70 | mg/mL |
| DPPC | 30-40 mg/mL | DPPC | ~32-35 | mg/mL |
| Cholesterol | 10-20 mg/mL | Cholesterol | ~16-17 | mg/mL |
| NaCl | 0.5%-5.0% | NaCl | ~1.5% | |
| Formulation C (pH 6.0-7.0) | | Formulation F (pH ~6.5) | | |
| Amikacin | 70-80 mg/mL | Amikacin | ~70 | mg/mL |
| DPPC | 35-40 mg/mL | DPPC | ~30-35 | mg/mL |
| Cholesterol | 15-20 mg/mL | Cholesterol | ~15-17 | mg/mL |

| | | | |
|---|---|---|---|
| NaCl | 0.5%-5.0% | NaCl | ~1.5% |

It should be noted that increasing aminoglycoside concentration alone may not result in a reduced dosing time. For example, in one embodiment, the lipid to drug ratio is fixed, and as amikacin concentration is increased (and therefore lipid concentration is increased, since the ratio of the two is fixed, for example at ˜0.7:1), the viscosity of the solution also increases, which slows nebulization time.

In one embodiment, prior to nebulization of the aminoglycoside formulation, about 70% to about 100% of the aminoglycoside present in the formulation is liposomal complexed. In a further embodiment, the aminoglycoside is an aminoglycoside. In even a further embodiment, the aminoglycoside is amikacin. In another embodiment, prior to nebulization, about 80% to about 99%, or about 85% to about 99%, or about 90% to about 99% or about 95% to about 99% or about 96% to about 99% of the aminoglycoside present in the formulation is liposomal complexed. In a further embodiment, the aminoglycoside is amikacin or tobramycin. In even a further embodiment, the aminoglycoside is amikacin. In another embodiment, prior to nebulization, about 98% of the aminoglycoside present in the formulation is liposomal complexed. In a further embodiment, the aminoglycoside is amikacin or tobramycin. In even a further embodiment, the aminoglycoside is amikacin.

In one embodiment, upon nebulization, about 20% to about 50% of the liposomal complexed aminoglycoside agent is released, due to shear stress on the liposomes. In a further embodiment, the aminoglycoside agent is an amikacin. In another embodiment, upon nebulization, about 25% to about 45%, or about 30% to about 40% of the liposomal complexed aminoglycoside agent is released, due to shear stress on the liposomes. In a further embodiment, the aminoglycoside agent is amikacin.

As provided herein, the present invention provides methods and systems for treatment of lung infections by inhalation of a liposomal aminoglycoside formulation via nebulization. The formulation, in one embodiment, is administered via a nebulizer, which provides an aerosol mist of the formulation for delivery to the lungs of a subject.

In one embodiment, the nebulizer described herein generates an aerosol (i.e., achieves a total output rate) of the aminoglycoside pharmaceutical formulation at a rate greater than about 0.53 g per minute, greater than about 0.54 g per minute, greater than about 0.55 g per minute, greater than about 0.58 g per minute, greater than about 0.60 g per minute, greater than about 0.65 g per minute or greater than about 0.70 g per minute. In another embodiment, the nebulizer described herein generates an aerosol (i.e., achieves a total output rate) of the aminoglycoside pharmaceutical formulation at about 0.53 g per minute to about 0.80 g per minute, at about 0.53 g per minute to about 0.70 g per minute, about 0.55 g per min to about 0.70 g per minute, about 0.53 g per minute to about 0.65 g per minute, or about 0.60 g per minute to about 0.70 g per minute. In yet another embodiment, the nebulizer described herein generates an acrosol (i.e., achieves a total output rate) of the aminoglycoside pharmaceutical formulation at about 0.53 g per minute to about 0.75 g per minute, about 0.55 g per min to about 0.75 g per minute, about 0.53 g per minute to about 0.65 g per minute, or about 0.60 g per minute to about 0.75 g per minute.

Upon nebulization, the liposomes in the pharmaceutical formulation leak drug. In one embodiment, the amount of liposomal complexed aminoglycoside post-nebulization is about 45% to about 85%, or about 50% to about 80% or about 51% to about 77%. These percentages are also referred to herein as “percent associated aminoglycoside post-nebulization”. As provided herein, in one embodiment, the liposomes comprise an aminoglycoside, e.g., amikacin. In one embodiment, the percent associated aminoglycoside post-nebulization is from about 60% to about 70%. In a further embodiment, the aminoglycoside is amikacin. In another embodiment, the percent associated aminoglycoside post-nebulization is about 67%, or about 65% to about 70%. In a further embodiment, the aminoglycoside is amikacin.

In one embodiment, the percent associated aminoglycoside post-nebulization is measured by reclaiming the aerosol from the air by condensation in a cold-trap, and the liquid is subsequently assayed for free and encapsulated aminoglycoside (associated aminoglycoside).

In one embodiment, the MMAD of the aerosol of the pharmaceutical formulation is less than 4.9 μm, less than 4.5 μm, less than 4.3 μm, less than 4.2 μm, less than 4.1 μm, less than 4.0 μm or less than 3.5 μm, as measured by the ACI at a gas flow rate of about 28 L/minute, or by the Next Generation Impactor NGI at a gas flow rate of about 15 L/minute.

In one embodiment, the MMAD of the aerosol of the pharmaceutical formulation is about 1.0 μm to about 4.2 μm, about 3.2 μm to about 4.2 μm, about 3.4 μm to about 4.0 μm, about 3.5 μm to about 4.0 μm or about 3.5 μm to about 4.2 μm, as measured by the ACI. In one embodiment, the MMAD of the aerosol of the pharmaceutical formulation is about 2.0 μm to about 4.9 μm, about 4.4 μm to about 4.9 μm, about 4.5 μm to about 4.9 μm, or about 4.6 μm to about 4.9 μm, as measured by the NGI.

In another embodiment, the nebulizer described herein generates an acrosol of the aminoglycoside pharmaceutical formulation at a rate greater than about 0.53 g per minute, greater than about 0.55 g per minute, or greater than about 0.60 g per minute or about 0.60 g per minute to about 0.70 g per minute. In a further embodiment, the FPF of the aerosol is greater than or equal to about 64%, as measured by the ACI, greater than or equal to about 70%, as measured by the ACI, greater than or equal to about 51%, as measured by the NGI, or greater than or equal to about 60%, as measured by the NGI.

In one embodiment, the system provided herein comprises a nebulizer selected from an electronic mesh nebulizer, pneumonic (jet) nebulizer, ultrasonic nebulizer, breath-enhanced nebulizer and breath-actuated nebulizer. In one embodiment, the nebulizer is portable.

The principle of operation of a pneumonic nebulizer is generally known to those of ordinary skill in the art and is described, e.g., in Respiratory Care, Vol. 45, No. 6, pp. 609-622 (2000). Briefly, a pressurized gas supply is used as the driving force for liquid atomization in a pneumatic nebulizer. Compressed gas is delivered, which causes a region of negative pressure. The solution to be aerosolized is then delivered into the gas stream and is sheared into a liquid film. This film is unstable and breaks into droplets because of surface tension forces. Smaller particles, i.e., particles with the MMAD and FPF properties described above, can then be formed by placing a baffle in the aerosol stream. In one pneumonic nebulizer embodiment, gas and solution is mixed prior to leaving the exit port (nozzle) and interacting with the baffle. In another embodiment, mixing does not take place until the liquid and gas leave the exit port (nozzle). In one embodiment, the gas is air, O₂ and/or CO₂.

In one embodiment, droplet size and output rate can be tailored in a pneumonic nebulizer. However, consideration should be paid to the formulation being nebulized, and whether the properties of the formulation (e.g., % associated aminoglycoside) are altered due to the modification of the nebulizer. For example, in one embodiment, the gas velocity and/or pharmaceutical formulation velocity is modified to achieve the output rate and droplet sizes of the present invention. Additionally or alternatively, the flow rate of the gas and/or solution can be tailored to achieve the droplet size and output rate of the invention. For example, an increase in gas velocity, in one embodiment, decreased droplet size. In one embodiment, the ratio of pharmaceutical formulation flow to gas flow is tailored to achieve the droplet size and output rate of the invention. In one embodiment, an increase in the ratio of liquid to gas flow increases particle size.

In one embodiment, a pneumonic nebulizer output rate is increased by increasing the fill volume in the liquid reservoir. Without wishing to be bound by theory, the increase in output rate may be due to a reduction of dead volume in the nebulizer. Nebulization time, in one embodiment, is reduced by increasing the flow to power the nebulizer. See, e.g., Clay et al. (1983). Lancet 2, pp. 592-594 and Hess et al. (1996). Chest 110, pp. 498-505.

In one embodiment, a reservoir bag is used to capture acrosol during the nebulization process, and the acrosol is subsequently provided to the subject via inhalation. In another embodiment, the nebulizer provided herein includes a valved open-vent design. In this embodiment, when the patient inhales through the nebulizer, nebulizer output is increased. During the expiratory phase, a one-way valve diverts patient flow away from the nebulizer chamber.

In one embodiment, the nebulizer provided herein is a continuous nebulizer. In other words, refilling the nebulizer with the pharmaceutical formulation while administering a dose is not needed. Rather, the nebulizer has at least an 8 mL capacity or at least a 10 mL capacity.

In one embodiment, a vibrating mesh nebulizer is used to deliver the aminoglycoside formulation of the invention to a patient in need thereof. In one embodiment, the nebulizer membrane vibrates at an ultrasonic frequency of about 100 kHz to about 250 kHz, about 110 kHz to about 200 kHz, about 110 kHz to about 200 kHz, about 110 kHz to about 150 kHz. In one embodiment, the nebulizer membrane vibrates at a frequency of about 117 kHz upon the application of an electric current.

In one embodiment, the nebulizer provided herein does not use an air compressor and therefore does not generate an air flow. In one embodiment, aerosol is produced by the aerosol head which enters the mixing chamber of the device. When the patient inhales, air enters the mixing chamber via one-way inhalation valves in the back of the mixing chamber and carries the aerosol through the mouthpiece to the patient. On exhalation, the patient's breath flows through the one-way exhalation valve on the mouthpiece of the device. In one embodiment, the nebulizer continues to generate aerosol into the mixing chamber which is then drawn in by the subject on the next breath—and this cycle continues until the nebulizer medication reservoir is empty.

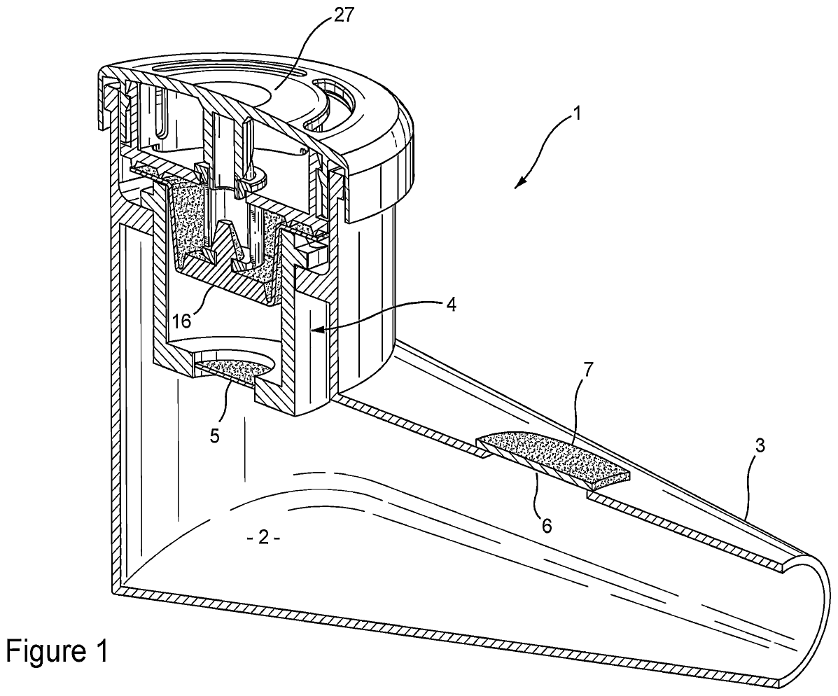

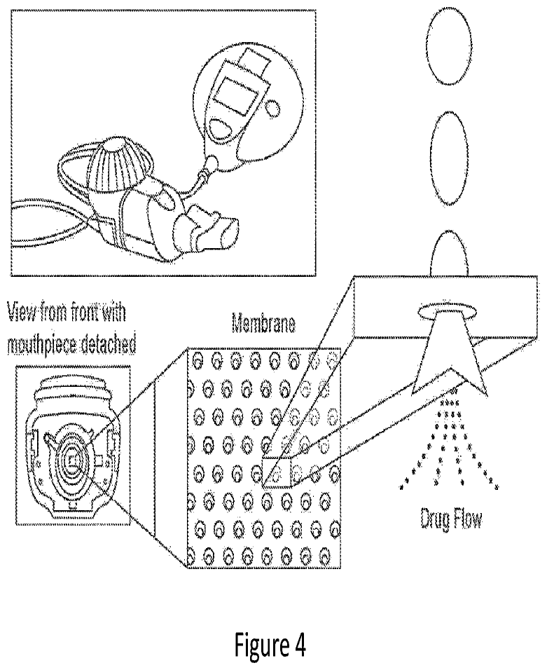

Although not limited thereto, the present invention, in one embodiment, is carried out with one of the aerosol generators (nebulizers) depicted in FIGS. 1, 2, 3 and 4. Additionally, the systems of the invention, in one embodiment, include a nebulizer described in European Patent Applications 11169080.6 and/or 10192385.2. These applications are incorporated by reference in their entireties.

FIG. 1 shows a therapeutic aerosol device 1 with a nebulizing chamber 2, a mouthpiece 3 and a membrane aerosol generator 4 with an oscillating membrane 5. The oscillating membrane may, for example, be brought to oscillation by annular piezo elements (not shown), examples of which are described in WO 1997/29851.

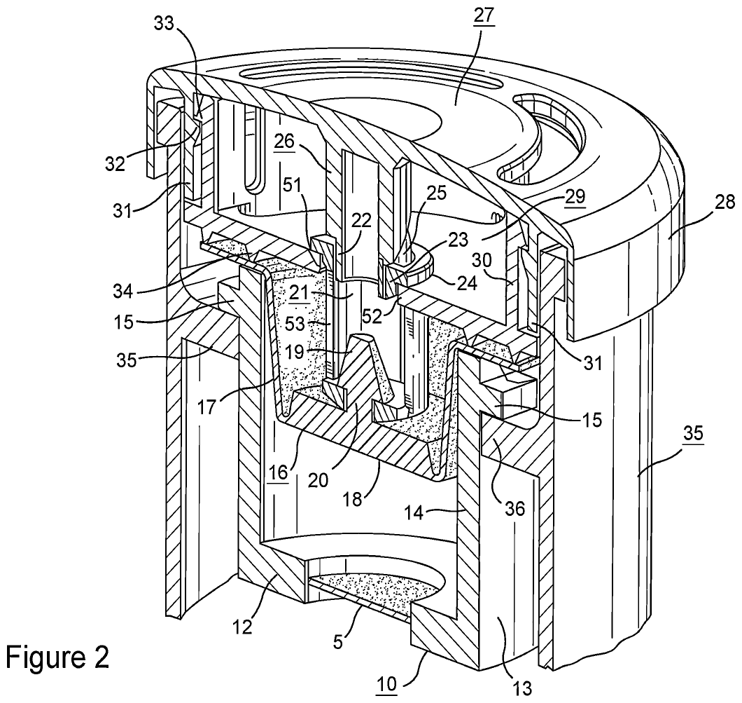

When in use, the pharmaceutical formulation is located on one side of the oscillating membrane 5, see FIGS. 1, 2 and 4, and this liquid is then transported through openings in the oscillating membrane 5 and emitted on the other side of the oscillating membrane 5, see bottom of FIG. 1, FIG. 2, as an acrosol into the nebulizing chamber 2. The patient is able to breathe in the aerosol present in the nebulizing chamber 2 at the mouthpiece 3.

The oscillating membrane 5 comprises a plurality of through holes. Droplets of the aminoglycoside formulation are generated when the aminoglycoside pharmaceutical formulation passes through the membrane. In one embodiment, the membrane is vibratable, a so called active electronic mesh nebulizer, for example the eFlow® nebulizer from PARI Pharma, HL100 nebulizer from Health and Life, or the Acroneb Go® from Acrogen (Novartis). In a further embodiment, the membrane vibrates at an ultrasonic frequency of about 100 kHz to about 150 kHz, about 110 kHz to about 140 kHz, or about 110 kHz to about 120 kHz. In a further embodiment, the membrane vibrates at a frequency of about 117 kHz upon the application of an electric current. In a further embodiment, the membrane is fixed and the a further part of the fluid reservoir or fluid supply is vibratable, a so called passive electronic mesh nebulizer, for example the MicroAir Electronic Nebulizer Model U22 from Omron or the I-Neb I-neb AAD Inhalation System from Philips Respironics.

In one embodiment, the length of the nozzle portion of the through holes formed in the membrane (e.g., vibratable membrane) influences the total output rate (TOR) of the aerosol generator. In particular, it has been found that the length of the nozzle portion is directly proportional to the total output rate, wherein the shorter the nozzle portion, the higher the TOR and vice versa.

In one embodiment, the nozzle portion is sufficiently short and small in diameter as compared to the upstream portion of the through hole. In a further embodiment, the length of the portions upstream of the nozzle portion within the through hole does not have a significant influence on the TOR.

In one embodiment, the length of the nozzle portion influences the geometric standard deviation (GSD) of the droplet size distribution of the aminoglycoside pharmaceutical formulation. Low GSDs characterize a narrow droplet size distribution (homogeneously sized droplets), which is advantageous for targeting aerosol to the respiratory system, for example for the treatment of bacterial infections (e.g., Pseudomonas or Mycobacteria) in cystic fibrosis patients, or the treatment of nontuberculosis Mycobacteria, bronchiectasis (e.g., the treatment of cystic fibrosis or non-cystic fibrosis patients), Pseudomonas or Mycobacteria in patients. That is, the longer the nozzle portion the lower the GSD. The average droplet size, in one embodiment is less than 5 μm, and has a GSD in a range of 1.0 to 2.2, or about 1.0 to about 2.2, or 1.5 to 2.2, or about 1.5 to about 2.2.

In one embodiment, as provided above, the system provided herein comprises a nebulizer which generates an aerosol of the aminoglycoside pharmaceutical formulation at a rate greater than about 0.53 g per minute, or greater than about 0.55 g per minute. In a further embodiment, the nebulizer comprises a vibratable membrane having a first side for being in contact with the fluid and an opposite second side, from which the droplets emerge.

The membrane, e.g., a stainless steel membrane, may be vibrated by means of a piezoelectric actuator or any other suitable means. The membrane has a plurality of through holes penetrating the membrane in an extension direction from the first side to the second side. The through holes may be formed as previously mentioned by a laser source, electroforming or any other suitable process. When the membrane is vibrating, the aminoglycoside pharmaceutical formulation passes the through holes from the first side to the second side to generate the aerosol at the second side. Each of the through holes, in one embodiment, comprises an entrance opening and an exit opening. In a further embodiment, each of the through holes comprises a nozzle portion extending from the exit opening over a portion of the through holes towards the entrance opening. The nozzle portion is defined by the continuous portion of the through hole in the extension direction comprising a smallest diameter of the through hole and bordered by a larger diameter of the through hole. In one embodiment, the larger diameter of the through hole is defined as that diameter that is closest to 3 times, about 3 times, 2 times, about 2 times, 1.5 times, or about 1.5 times, the smallest diameter.

The smallest diameter of the through hole, in one embodiment, is the diameter of the exit opening. In another embodiment, the smallest diameter of the through hole is a diameter about 0.5×, about 0.6×, about 0.7×, about 0.8× or about 0.9× the diameter of the exit opening.

In one embodiment, the nebulizer provided herein comprises through holes in which the ratio of the total length of at least one of the through holes in the extension direction to the length of the respective nozzle portion of the through hole in the extension direction is at least 4, or at least about 4, or at least 4.5, or at least about 4.5, or at least 5, or at least about 5, or greater than about 5. In another embodiment, the nebulizer provided herein comprises through holes in which the ratio of the total length of the majority of through holes in the extension direction to the length of the respective nozzle portion of the through holes in the extension direction is at least 4, or at least about 4, or at least 4.5, or at least about 4.5, or at least 5, or at least about 5, or greater than about 5.

The extension ratios set forth above provide, in one embodiment, an increased total output rate, as compared to previously known nebulizers, and also provides a sufficient GSD. The ratio configurations, in one embodiment, achieve shorter application periods, leading to greater comfort for the patient and effectiveness of the aminoglycoside compound. This is particularly advantageous if the aminoglycoside compound in the formulation, due to its properties, is prepared at a low concentration, and therefore, a greater volume of the aminoglycoside pharmaceutical formulation must be administered in an acceptable time, e.g., one dosing session.

According to one embodiment, the nozzle portion terminates flush with the second side. Therefore, the length of the nozzle portion, in one embodiment, is defined as that portion starting from the second side towards the first side up to and bordered by the diameter that it is closest to about triple, about twice, about 2.5×, or about 1.5× the smallest diameter. The smallest diameter, in this embodiment, is the diameter of the exit opening.

In one embodiment, the smallest diameter (i.e., one border of the nozzle portion) is located at the end of the nozzle portion in the extension direction adjacent to the second side. In one embodiment, the larger diameter of the through hole, located at the other border of the nozzle portion, is located upstream of the smallest diameter in the direction in which the fluid passes the plurality of through holes during operation.

According to one embodiment, the smallest diameter is smaller than about 4.5 μm, smaller than about 4.0 μm, smaller than about 3.5 μm, or smaller than about 3.0 μm.

In one embodiment, the total length of at least one through hole in the extension direction is at least about 50 μm, at least about 60 μm, at least about 70 μm, or at least about 80 μm. In a further embodiment, the total length of at least one of the plurality of through holes is at least about 90 μm. In one embodiment, the total length of a majority of the plurality of through holes in the extension direction is at least about 50 μm, at least about 60 μm, at least about 70 μm, or at least about 80 μm. In a further embodiment, the total length of a majority of the plurality of through holes is at least about 90 μm.

The length of the nozzle portion, in one embodiment, is less than about 25 μm, less than about 20 μm or less than about 15 μm.

According to one embodiment, the through holes are laser-drilled through holes formed in at least two stages, one stage forming the nozzle portion and the remaining stage(s) forming the remainder of the through holes.

In another embodiment, the manufacturing methods used lead to a nozzle portion which is substantially cylindrical or conical with a tolerance of less than +100% of the smallest diameter, less than +75% of the smallest diameter, less than +50% of the smallest diameter, less than +30% of the smallest diameter, less than +25% of the smallest diameter, or less than +15% of the smallest diameter.

Alternatively or additionally, the through holes are formed in an electroforming process. In one embodiment, the through holes have a first funnel-shaped portion at the first side and a second funnel-shaped portion at the second side with the nozzle portion in-between the first and the second funnel-shaped portions and defined between the exit opening and the larger diameter. In this instance, the total length of the through holes may as well be defined by the distances from the first side to the exit opening (smallest diameter) only.

In addition, the total output rate (TOR) may be further increased by increasing the number of through holes provided in the membrane. In one embodiment, an increase in number of through holes is achieved by increasing the active perforated surface of the membrane and maintaining the distance of the through holes relative to each other at the same level. In another embodiment, the number of through holes is increased by reducing the distance of the through holes relative to each other and maintaining the active area of the membrane. In addition, a combination of the above strategies may be used.

In one embodiment, the total output rate of the nebulizer described herein is increased by increasing the density of through holes in the membrane. In one embodiment, the average distance between through holes is about 70 μm, or about 60 μm, or about 50 μm.

In one embodiment, the membrane comprises between about 200 and about 8,000 through holes, between about 1,000 and about 6,000 through holes, between about 2,000 and about 5,000 through holes or about 2,000 and about 4,000 through holes. In one embodiment, the number of through holes described above increases the TOR, and the TOR is increased regardless of whether the nozzle parameters are implemented as described above. In one embodiment, the nebulizer provided herein comprises about 3,000 through holes. In a further embodiment, the through holes are located in a hexagonal array, e.g., at about the center of the membrane (e.g., stainless steel membrane). In a further embodiment, the average distance between through holes is about 70 μm.

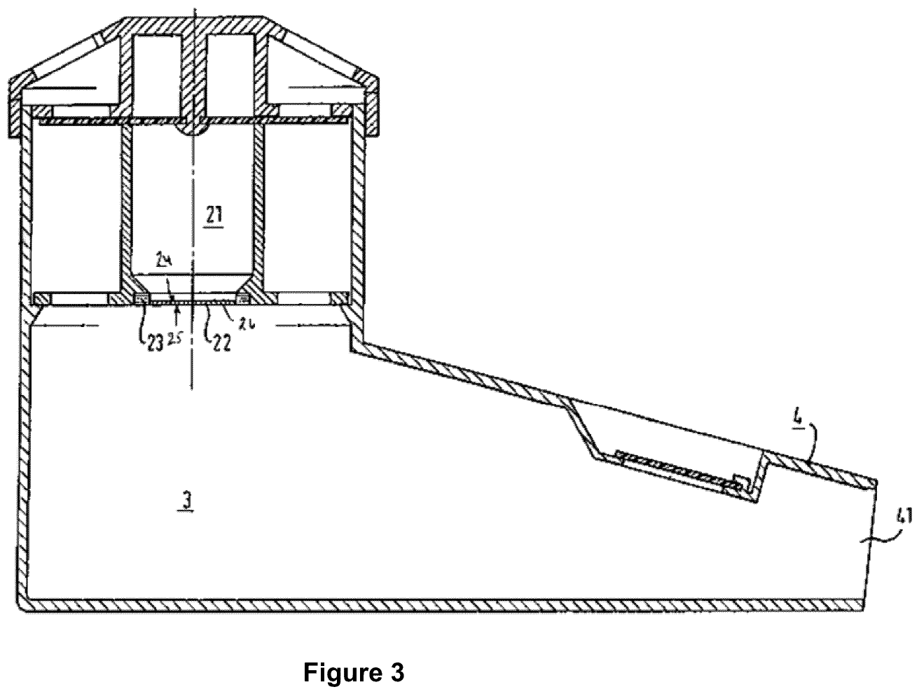

FIG. 3 shows an aerosol generator (nebulizer) as disclosed in WO 2001/032246, which is hereby incorporated by reference in its entirety. The aerosol generator comprises a fluid reservoir 21 to contain the pharmaceutical formulation, to be emitted into the mixing chamber 3 in the form of an acrosol and to be inhaled by means of the mouth piece 4 through the opening 41.

The aerosol generator comprises a vibratable membrane 22 vibrated by means of a piezoelectric actuator 23. The vibratable membrane 22 has a first side 24 facing the fluid container 21 and a second opposite side 25 facing the mixing chamber 3. In use, the first side 24 of the vibratable membrane 22 is in contact with the fluid contained in the fluid container 21. A plurality of through holes 26 penetrating the membrane from the first side 24 to the second side 25 are provided in the membrane 22. In use, the fluid passes from the fluid container 21 through the through holes 26 from the first 24 to the second side 25 when the membrane 22 is vibrated for generating the aerosol at the second side 25 and emitting it into the mixing chamber 3. This aerosol may then be drawn by inhalation of a patient from the mixing chamber 3 via the mouth piece 4 and its inhalation opening 41.

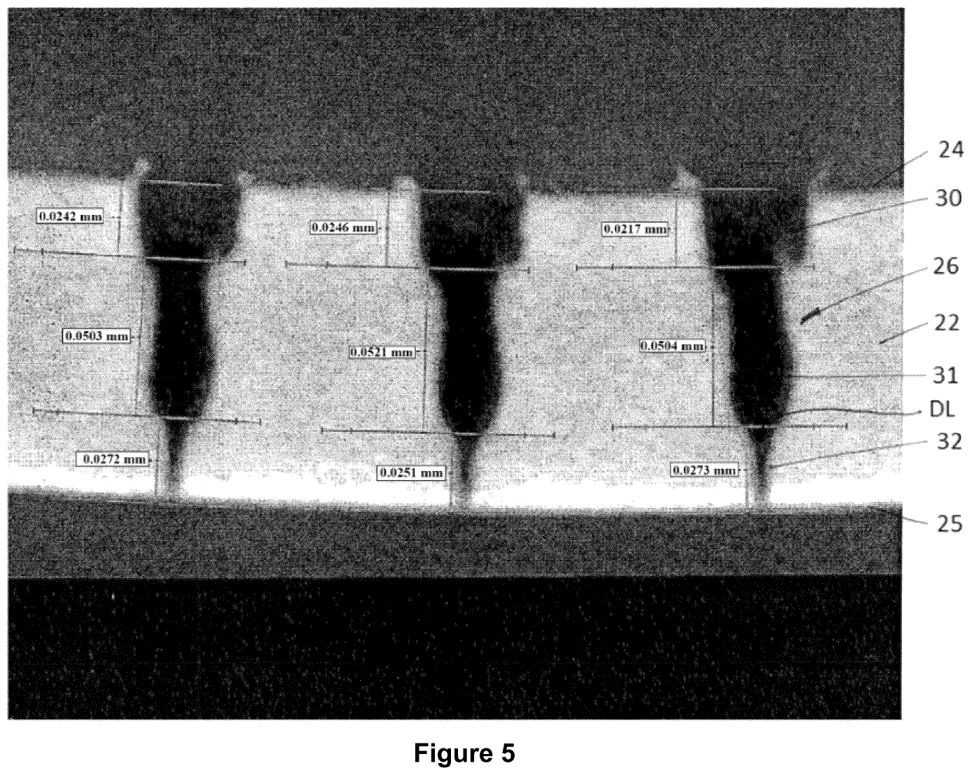

FIG. 5 shows a cross-sectional computed tomography scan showing three of the through holes 26 of such a vibratable membrane 22. The through holes 26 of this particular embodiment are formed by laser drilling using three stages of different process parameters, respectively. In a first stage, the portion 30 is formed. In a second stage the portion 31 is formed and in a third stage the nozzle portion 32 is formed. In this particular embodiment, the length of the nozzle portion 32 is about 26 μm, whereas the portion 31 has a length of about 51 μm. The first portion 30 has a length of about 24.5 μm. As a result, the total length of each through hole is the sum of the length of the portion 30, the portion 31 and the nozzle portion 32, that is in this particular example, about 101.5 μm. Thus, the ratio of the total length of each through hole 26 in the extension direction E to the length of a respective one of the nozzle portions 32 in the extension direction E is approximately 3.9.

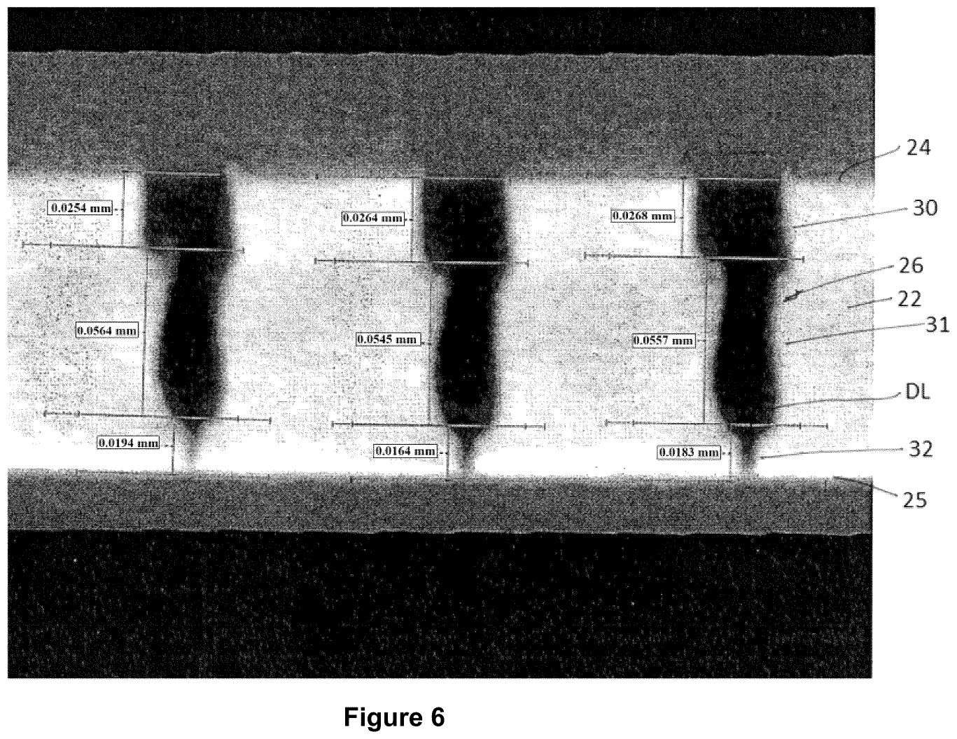

In the embodiment in FIG. 6, the first portion 30 has a length of about 27 μm, the portion 31 a length of about 55 μm and a nozzle portion a length of about 19 μm. As a result, the total length of the through hole 26 is about 101 μm. Thus, the ratio of the total length of the through hole 26 to the length of the corresponding nozzle portion 32 in this embodiment is approximately 5.3.

Both the vibratable membranes in FIGS. 5 and 6 were manufactured with 6,000 through holes 26. The below table (Table 3) indicates the mass median diameter (MMD), as determined by laser diffraction, of the particles emitted at the second side of the membrane, the time required for completely emitting a certain amount of liquid (Nebulization time) as well as the TOR. The tests were performed with a liposomal formulation of amikacin.

TABLE 3
Properties of nebulizer membranes.

| | | Nebulization | | # of |
|---|---|---|---|---|
| | MMD | time | TOR | through |
| Membrane | (μm) | (min) | (g/min.) | holes 26 |
| 1 (shown in FIG. 5 with a | 4.2 | 14.6 | 0.57 | 6,000 |
| nozzle portion of 26 μm) | | | | |
| 2 (shown in FIG. 6 with a | 4.3 | 9.3 | 0.89 | 6,000 |
| nozzle portion of 19 μm) | | | | |
| 3 (similar to FIG. 6) | 4.4 | 13.4 | 0.62 | 3,000 |
| 4 (similar to FIG. 6, | 4.4 | 11.9 | 0.7 | 3,000 |
| nozzle shorter than | | | | |
| membrane 3) | | | | |

Table 3 shows that the membrane 2 with the shorter nozzle portion provides for an increased TOR and a reduced nebulization time by 5.3 minutes, which is approximately 36% less as compared to the membrane 1. Table 3 also shows that the MMD did not vary significantly for each membrane tested. This is in contrast to the differences in TOR observed for each membrane. Thus, in one embodiment, the nebulization time for the nebulizer described herein is reduced significantly as compared to prior art nebulizers, without affecting the droplet size, as measured by MMD.

In addition to the membrane shown in FIGS. 5 and 6, membranes were manufactured having the nozzle portion further reduced, and with 3,000 through holes 26 (membranes 3 and 4, Table 3). In particular, a membrane 3 was laser-drilled with a shorter nozzle portion, whereas membrane 4 was manufactured using a shorter nozzle portion than membrane 3. Table 3 indicates that even with 3,000 holes (membrane 3 and 4) a reduction in the length of the nozzle portion results in an increased TOR compared to membrane 1 with 6,000 holes. The comparison of the membrane 3 and 4 as compared to the membrane 2 further shows that a combination of a higher number of holes (6,000 as compared to 3,000) and a reduced length of the nozzle portion increases the TOR for the nebulizer.

In one embodiment, it is advantageous to use a laser drilling process as compared to electroforming for manufacturing the through holes. The through holes shown in FIGS. 5 and 6, manufactured by laser drilling, are substantially cylindrical or conical as compared to the funnel-shaped entrance and exit of electro-formed through holes, e.g., as disclosed in WO 01/18280. The vibration of the membrane, that is its vibration velocity, may be transferred to the pharmaceutical formulation over a larger area by means of friction when the through holes are substantially cylindrical or conical as compared to the funnel-shaped entrance and exit of electro-formed through holes. The pharmaceutical formulation, because of its own inertia, is then ejected from the exit openings of the through holes resulting in liquid jets collapsing to form the aerosol. Without wishing to be bound by theory, it is thought that because an electro-formed membrane comprises extremely bent surfaces of the through holes, the surface or area for transferring the energy from the membrane to the liquid is reduced.

However, the present invention may also be implemented in electro-formed membranes, wherein the nozzle portion is defined by the continuous portion of the through hole in the extension direction starting from the smallest diameter of the through hole towards the first side until it reaches a diameter 2× or 3× of the smallest diameter of the hole. In one embodiment, the total length of the through hole is measured from the smallest diameter to the first side.

Referring again to FIG. 1, so that the patient does not have to remove or to put down the therapeutic device from his mouth after inhaling the aerosol, the mouthpiece 3 has an opening 6 sealed by an elastic valve element 7 (exhalation valve). If the patient exhales into the mouthpiece 3 and hence into the nebulizing chamber 2, the elastic valve element 7 opens so that the exhaled air is able to escape from the interior of the therapeutic aerosol. On inhalation, ambient air flows through the nebulizing chamber 2. The nebulizing chamber 2 has an opening sealed (not shown) by a further elastic valve element (inhalation valve). If the patient inhales through the mouthpiece 3 and sucks from the nebulizing chamber 2, the elastic valve clement opens so that the ambient air is able to enter into the nebulizing chamber and mixed with the aerosol and leave the interior of the nebulizing chamber 2 to be inhaled. Further description of this process is provided in U.S. Pat. No. 6,962,151, which is incorporated by reference in its entirety for all purposes.

The nebulizer shown in FIG. 2 comprises a cylindrical storage vessel 10 to supply a liquid that is fed to the membrane 5. As shown in FIG. 2, the oscillating membrane 5 may be arranged in an end wall 12 of the cylindrical liquid reservoir 10 to ensure that the liquid poured into the liquid reservoir comes into direct contact with the membrane 5 when the aerosol generator is held in the position shown in FIG. 1. However, other methods may also be used to feed the liquid to the oscillating membrane without any change being necessary to the design of the device according to the invention for the generation of a negative pressure in the liquid reservoir.

On the side facing the end wall 12, the cylindrical liquid container 10 is open. The opening is used to pour the liquid into the liquid reservoir 10. Slightly below the opening on the external surface 13 of the peripheral wall 14 there is a projection 15 which serves as a support when the liquid container is inserted in an appropriately embodied opening in a housing 35.

The open end of the liquid container 10 is closed by a flexible sealing element 16. The sealing element 16 lies on the end of the peripheral wall 14 of the liquid container 10 and extends in a pot-shaped way into the interior of the liquid container 10 whereby a conically running wall section 17 is formed in the sealing element 16 and closed off by a flat wall section 18 of the scaling element 16. As discussed further below, forces act via the flat wall section 18 on the sealing element 16 and so in one embodiment the flat wall section 18 is thicker than the other sections of the sealing element 16. On the perimeter of the flat wall section 18, there is a distance to the conical wall section 17 so that the conical wall section 17 may be folded when the flat wall section 18 is moved upwards, relative to the representation in FIG. 2.

On the side of the flat wall section 18 facing away from the interior of the liquid container, there is a projection comprising a truncated cone section 19 and a cylindrical section 20. This design enables the projection to be introduced and latched into an opening adapted to match the cylindrical section since the flexible material of the sealing element 16 permits the deformation of the truncated cone section 19.

In one embodiment, the aerosol generator 4 comprises a slidable sleeve 21 equipped with an opening of this type which is substantially a hollow cylinder open on one side. The opening for the attachment of the sealing element 16 is embodied in an end wall of the slidable sleeve 21. When the truncated cone 19 has latched into place, the end wall of the slidable sleeve 21 containing the opening lies on the flat sealing element wall section 18. The latching of the truncated cone 19 into the slidable sleeve enables forces to be transmitted from the slidable sleeve 21 onto the flat wall section 18 of the sealing element 16 so that the sealing section 18 follows the movements of the slidable sleeve 21 in the direction of the central longitudinal axis of the liquid container 10.

In a generalized form, the slidable sleeve 21 may be seen as a slidable element, which may, for example, also be implemented as a slidable rod which may be stuck-on or inserted in a drill hole. Characteristic of the slidable element 21 is the fact that it may be used to apply a substantially linearly directed force onto the flat wall element 18 of the sealing element 16. Overall, the decisive factor for the mode of operation of the aerosol generator according to the invention is the fact that a slidable element transmits a linear movement onto the sealing element so that an increase in volume occurs within the liquid reservoir 10. Since the liquid reservoir 10 is otherwise gas-tight, this causes a negative pressure to be generated in the liquid reservoir 10.

The sealing element 16 and the slidable element 21 may be produced in one piece, i.e., in one operation, but from different materials. The production technology for this is available so that a one-piece component for the nebulizer is created, e.g., in a fully automatic production step.

In one embodiment, the slidable sleeve 21 is open on the end facing the drill hole for the truncated cone but at least two diametrically opposite lugs 22 and 23 protrude radially into the interior of the slidable sleeve 21. A collar 24 encircling the slidable sleeve extends radially outwards. While the collar 24 is used as a support for the slidable sleeve 21 in the position shown in FIG. 5, the projections 22 and 23 protruding into the interior of the slidable sleeve 21 are used to absorb the forces acting on the slidable sleeve 21 in particular parallel to the central longitudinal axis. In one embodiment, these forces are generated by means of two spiral grooves 25 which are located on the outside of the peripheral wall of a rotary sleeve 26.

In one embodiment, the nebulizer may be implemented with one of the projections 22 or 23 and one groove 25. In a further embodiment, a uniformly distributed arrangement of two or more projections and a corresponding number of grooves is provided.

In one embodiment, the rotary sleeve 26 is also a cylinder open on one side whereby the open end is arranged in the slidable sleeve 21 and is hence facing the truncated cone 19 enabling the truncated cone 19 to penetrate the rotary sleeve 26. In addition, the rotary sleeve 26 is arranged in the slidable sleeve 21 in such a way that the projections 22 and 23 lie in the spiral grooves 25. The inclination of the spiral groove 25 is designed so that, when the rotary sleeve 26 is rotated in relation to the slidable sleeve 21, the projections 22 and 23 slide along the spiral grooves 25 causing a force directed parallel to the central longitudinal axis to be exerted on the sliding projections 22 and 23 and hence on the slidable sleeve 21. This force displaces the slidable sleeve 21 in the direction of the central longitudinal axis so that the sealing element 16 which is latched into the slidable sleeve's drill hole by means of the truncated cone is also substantially displaced parallel to the central longitudinal axis.

The displacement of the sealing element 16 in the direction of the central longitudinal axis of the liquid container 10 generates a negative pressure in the liquid container 10, determined inter alia by the distance by which the slidable sleeve 21 is displaced in the direction of the central longitudinal axis. The displacement causes the initial volume V_{RI} of the gas-tight liquid container 10 to increase to the volume V_{RN} and thereby a negative pressure to be generated. The displacement is in turn defined by the design of the spiral grooves 25 in the rotary sleeve 26. In this way, the aerosol generator according to the invention ensures that the negative pressure in the liquid reservoir 10 may be generated in the relevant areas by means of simple structural measures.

To ensure that the forces to be applied to generate the negative pressure when handling the device remain low, the rotary sleeve 26 is embodied in one piece with a handle 27 whose size is selected to enable the user to rotate the handle 27, and hence the rotary sleeve 26, manually without great effort. The handle 27 substantially has the shape of a flat cylinder or truncated cone which is open on one side so that a peripheral gripping area 28 is formed on the external periphery of the handle 27 which is touched by the user's hand to turn the handle 27.

Due to the design of the spiral grooves 25 and the overall comparatively short distance to be travelled by the slidable sleeve 21 in the longitudinal direction to generate a sufficient negative pressure, in one embodiment, it is sufficient to turn the handle 27 and hence the rotary sleeve 26 through a comparatively small angle of rotation. In one embodiment, the angle of rotation lies within a range from 45 to 360 degrees. This embodiment allows for the case of handling of the device according to the invention and the therapeutic aerosol generator equipped therewith.

In order to create a unit which may be operated simply and uniformly from the slidable sleeve 21 and the rotary sleeve 26 including the handle 27, in one embodiment, the aerosol generator described here has a bearing sleeve 29 for bearing the slidable sleeve 21, which substantially comprises a flat cylinder open on one side. The diameter of the peripheral wall 30 of the bearing sleeve 29 is smaller than the internal diameter of the handle 27 and, in the example of an embodiment described, is aligned on the internal diameter of a cylindrical latching ring 31 which is provided concentrically to the gripping area 28 of the handle 27 but with a smaller diameter on the side of the handle 27 on which the rotary sleeve 26 is also arranged. Embodied on the side of the cylindrical latching ring 31 facing the rotary sleeve is a peripheral latching edge 32 which may be brought into engagement with latching lugs 33 situated at intervals on the peripheral wall 30 of the bearing sleeve 29. This allows the handle 27 to be located on the bearing sleeve 29 whereby, as shown in FIG. 5, the handle 27 is placed on the open end of the bearing sleeve 29 and the latching edge 32 is interlatched with the latching lugs 33.

To hold the slidable sleeve 21, an opening is provided in the centre of the sealed end of the bearing sleeve 29 in which the slidable sleeve 21 is arranged, as may be identified in FIG. 2. The collar 24 of the slidable sleeve 21 lies in the position shown in FIG. 2 on the surface of the end wall of the bearing sleeve 29 facing the handle. Extending into the bearing opening are two diametrically opposite projections 51 and 52, which protrude into two longitudinal grooves 53 and 54 on the peripheral surface of the slidable sleeve 21. The longitudinal grooves 53 and 54 run parallel to the longitudinal axis of the slidable sleeve 21. The guide projections 51 and 52 and the longitudinal grooves 53 and 54 provide anti-rotation locking for the slidable sleeve 21 so that the rotational movement of the rotary sleeve 26 results not in rotation but in the linear displacement of the slidable sleeve 21. As is evident from FIG. 2, this ensures that the slidable sleeve 21 is held in the combination of the handle 27 and the bearing sleeve 29 in an axially displaceable way but locked against rotation. If the handle 27 is rotated in relation to the bearing sleeve 29, the rotary sleeve 26 also rotates in relation to the slidable sleeve 21 whereby the sliding projections 22 and 23 move along the spiral grooves 25. This causes the slidable sleeve 21 to be displaced in an axial direction in the opening of the bearing sleeve 29.

It is possible to dispense with the guide projections 51 and 52 in the bearing opening and the longitudinal grooves 53 and 54 in the slidable sleeve 21. In one embodiment, the guide projections 51 and 52 and the longitudinal grooves 53 and 54 are not present in the aerosol generator, and the truncated cone 19, the cylinder sections 20 of the sealing elements 16 and the large-area support for the slidable sleeve 21 holding the truncated cone on the flat sealing element section 18 achieves anti-rotation locking of the slidable sleeve 21 by means of friction. In a further embodiment, the sealing element 16 is fixed so it is unable to rotate in relation to the bearing sleeve 29.

In one embodiment, provided on the surface of the sealed end of the bearing sleeve 19 facing away from the handle, is an annular first sealing lip 34 concentric to the opening holding the slidable sleeve. The diameter of the first sealing lip 34 corresponds to the diameter of the peripheral wall 14 of the liquid container 10. As provided in FIG. 2, this ensures that the first sealing lip 34 presses the sealing element 16 on the end of the peripheral wall against the liquid reservoir 10 in such a way that the liquid reservoir 10 is sealed. In addition, the first sealing lip 34 may also fix the sealing element 16 so that it is unable to rotate in relation to the liquid reservoir 10 and the bearing sleeve 29. In one embodiment, excessive force need not be applied in order to ensure that the aforesaid components of the device are unable to rotate in relation to each other.

In one embodiment, the forces required are generated at least to some extent by means of an interaction between the handle 27 and the housing 35 in which the pharmaceutical formulation reservoir is embodied as one piece or in which the pharmaceutical formulation (liquid) reservoir 10 is inserted as shown in FIG. 2. In this case, the pharmaceutical formulation reservoir 10 inserted in the casing with the peripheral projection 15 lies at intervals on a support 36 in the housing 35 which extends radially into the interior of the housing 35. This allows the liquid reservoir 10 to be easily removed from the housing 35 for purposes of cleaning. In the embodiment shown in FIG. 2, support is only provided at intervals, and therefore, openings are provided for ambient air when the patient inhales, described in more detail below.

Identifiable in FIG. 2 is the rotary lock, which is implemented by means of the handle 27 on the one hand and the housing 35 on the other. Shown are the locking projections 62 and 63 on the housing 35. However, there are no special requirements with regard to the design of the rotary lock as far as the device according to invention is concerned for the generation of the negative pressure in the liquid reservoir 10.

In one embodiment, the liquid reservoir 10 is configured to have a volume V_{RN} of at least at least 16 mL, at least about 16 mL, at least 18 mL, at least about 18 mL, at least 20 mL or at least about 20 mL so that when for example, an amount of 8 mL of liquid (e.g., aminoglycoside pharmaceutical formulation) to be emitted in the form of an aerosol is contained in (filled or poured into) the liquid reservoir 10, an air cushion of 8 mL or about 8 mL is provided. That is, the ratio of the volume V_{RN} to the initial volume of liquid V_{L} within the liquid reservoir 10 is at least 2.0 and the ratio between the volume V_{A} of a gas and V_{L} of the liquid is at least 1.0. It has been shown that a liquid reservoir having a volume V_{RN} of about 15.5 mL, about 19.5 mL and about 22.5 mL are efficient, and that efficiency increases with the increase in V_{RN}.

In one embodiment, the ratio between V_{RN} and V_{L} is at least 2.0, at least about 2.0, at least 2.4, at least about 2.4, at least 2.8 or at least about 2.8. In one embodiment, the ratio between V_{A} and V_{L} is at least 1.0, at least 1.2, at least 1.4, at least 1.6 or at least 1.8. In another embodiment, the ratio between V_{A} and V_{L} is at least about 1.0, at least about 1.2, at least about 1.4, at least about 1.6 or at least about 1.8.

The volume of the air cushion, in one embodiment, is at least 2 mL, at least about 2mL, at least 4 mL, at least about 4 mL, is at least 6 mL, at least about 6 mL, at least 8 mL, at least about 8 mL, at least 10 mL, at least about 10 mL, at least 11 mL, at least about 11 mL, at least 12 mL, at least about 12 mL, at least 13 mL, at least about 13 mL, at least 14 mL or at least about 14 mL. In one embodiment, the volume of the air cushion is at least about 11 mL or at least about 14 mL. In one embodiment, the volume of the air cushion is from about 6 mL to about 15 mL, and the ratio between V_{RN} and V_{L} is at least about 2.0 to at least about 3.0. In a further embodiment, the between V_{RN} and V_{L} is at least about 2.0 to about at least about 2.8.

The volume of the air cushion, in one embodiment, is about 2mL, about 4 mL, about 6 mL, about 8 mL, about 10 mL, about 11 mL, about 12 mL, about 13 mL, or about 14 mL.

In one embodiment, the ratio of the volume V_{RN} to the initial volume of liquid V_{L} is at least 2.0. Theoretically an unlimited enlargement of the increased volume V_{RN} of the liquid reservoir 10 will result in a nearly stable negative pressure range. In one embodiment, the ratio of the volume V_{RN} to the initial volume of liquid V_{L} is within the range between 2.0 and 4.0 and in a further embodiment is between 2.4 and 3.2. Two examples of the ratio ranges (V_{RN}/V_{L}) for different initial volume of liquid V_{L} between 4 mL and 8 mL are given in Table 4, below.

TABLE 4
Nebulizer Reservoir specifications

| V_{L} | V_{RN} | Ratio (V_{RN}/V_{L}) |
|---|---|---|
| 4 mL | 8.0-16.0 | 2.0-4.0 |
| 4 mL | 9.5-12.8 | 2.4-3.2 |
| 5 mL | 10.0-20.0 | 2.0-4.0 |
| 5 mL | 12.0-16.0 | 2.4-3.2 |
| 6 mL | 12.0-24.0 | 2.0-4.0 |
| 6 mL | 14.5-19.2 | 2.4-3.2 |
| 8 mL | 16.0-32.0 | 2.0-4.0 |
| 8 mL | 19.5-25.6 | 2.4-3.2 |

The systems provided herein may be used to treat a variety of pulmonary infections in subjects in need thereof. Among the pulmonary infections (such as in cystic fibrosis patients) that can be treated with the methods of the invention are gram negative infections. In one embodiment, infections caused by the following bacteria are treatable with the systems and formulations provided herein: Pseudomonas (e.g., P. aeruginosa, P. paucimobilis, P. putida, P. fluorescens, and P. acidovorans), Burkholderia (e.g., B. pseudomallei, B. cepacia, B. cepacia complex, B. dolosa, B. fungorum, B. gladioli, B. multivorans, B. vietnamiensis, B. pseudomallei, B. ambifaria, B. andropogonis, B. anthina, B. brasilensis, B. caledonica, B. caribensis, B. caryophylli), Staphylococcus (e.g., S. aureus, S. auricularis, S. carnosus, S. epidermidis, S. lugdunensis), Methicillin-resistant Staphylococcus aureus (MRSA), Streptococcus (e.g., Streptococcus pneumoniae), Escherichia coli, Klebsiella, Enterobacter, Serratia, Haemophilus, Yersinia pestis, Mycobacterium, nontuberculous Mycobacterium (e.g., M. avium, M. avium subsp. hominissuis (MAH), M. abscessus, M. chelonae, M. bolletii, M. kansasii, M. ulcerans, M. avium, M. avium complex (MAC) (M. avium and M. intracellulare), M. conspicuum, M. kansasii, M. peregrinum, M. immunogenum, M. xenopi, M. marinum, M. malmoense, M. marinum, M. mucogenicum, M. nonchromogenicum, M. scrofulaceum, M. simiae, M. smegmatis, M. szulgai, M. terrae, M. terrae complex, M. haemophilusenavense, M. asiaticum, M. shimoidei, M. gordonae, M. nonchromogenicum, M. triplex, M. lentiflavum, M. celatum, M. fortuitum, M. fortuitum complex (M. fortuitum and M. chelonae)).

In one embodiment, the systems described herein are used to treat an infection caused by a nontuberculous mycobacterial infection. In one embodiment, the systems described herein are used to treat an infection caused by Pseudomonas aeruginosa, Mycobacterium abscessus, Mycobaterium avium or M. avium complex. In a further embodiment, a patient with cystic fibrosis is treated for a Pseudomonas aeruginosa, Mycobacterium abscessus, Mycobaterium avium, or Mycobaterium avium complex infection with one or more of the systems described herein. In even a further embodiment, the Mycobaterium avium infection is Mycobacterium avium subsp. hominissuis.

In one embodiment, a patient with cystic fibrosis is treated for a pulmonary infection with one of the systems provided herein. In a further embodiment, the pulmonary infection is a Pseudomonas infection. In yet a further embodiment, the Pseudomonas infection is P. aeruginosa. In a further embodiment, the aminoglycoside in the system is amikacin.

In one embodiment, the system provided herein is used for the treatment or prophylaxis of Pseudomonas aeruginosa, Mycobacterium abscessus, Mycobaterium avium or Mycobaterium avium complex lung infection in a cystic fibrosis patient or a non-cystic fibrosis patient. In a further embodiment, the system provided herein comprises a liposomal aminoglycoside formulation. In a further embodiment, the aminoglycoside is selected from amikacin, apramycin, arbekacin, astromicin, capreomycin, dibekacin, framycetin, gentamicin, hygromycin B, isepamicin, kanamycin, neomycin, netilmicin, paromomycin, rhodestreptomycin, ribostamycin, sisomicin, spectinomycin, streptomycin, tobramycin, verdamicin or a combination thereof. In even a further embodiment, the aminoglycoside is amikacin, e.g., amikacin sulfate.

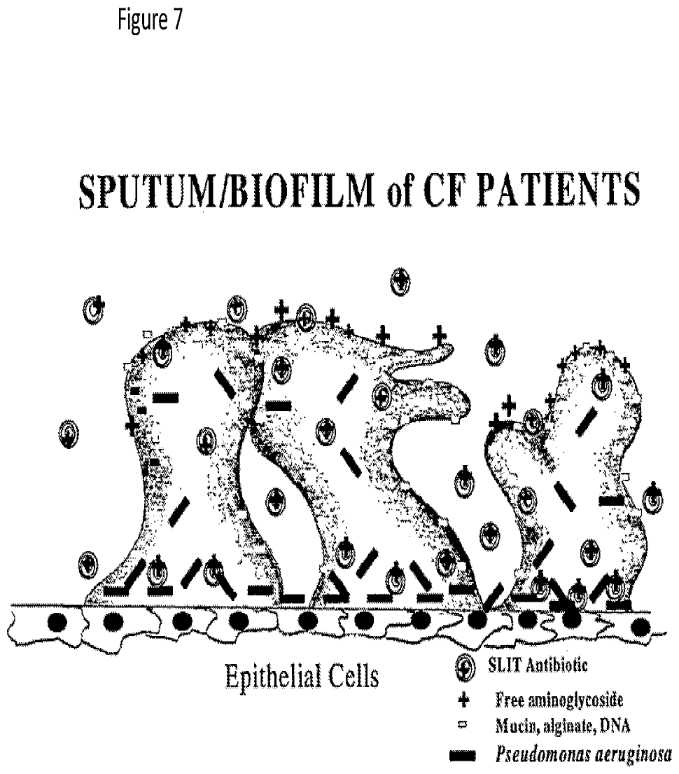

An obstacle to treating infectious diseases such as Pseudomonas aeruginosa, the leading cause of chronic illness in cystic fibrosis patients is drug penetration within the sputum/biofilm barrier on epithelial cells (FIG. 7). In FIG. 7, the donut shapes represent liposomal/complexed aminoglycoside, the “+” symbol represents free aminoglycoside, the “−” symbol mucin, alginate and DNA, and the solid bar symbol represents Pseudomonas aeruginosa. This barrier comprises both colonized and planktonic P. aeruginosa embedded in alginate or exopolysaccharides from bacteria, as well as DNA from damaged leukocytes, and mucin from lung epithelial cells, all possessing a net negative charge. The negative charge binds up and prevents penetration of positively charged drugs such as aminoglycosides, rendering them biologically ineffective (Mendelman et al., 1985). Without wishing to be bound by theory, entrapment of aminoglycosides within liposomes or lipid complexes shields or partially shields the aminoglycosides from non-specific binding to the sputum/biofilm, allowing for liposomes or lipid complexes (with entrapped aminoglycoside) to penetrate (FIG. 7).

In another embodiment, a patient is treated for nontuberculous Mycobacteria lung infection with one of the systems provided herein. In a further embodiment, the system provided herein comprises a liposomal amikacin formulation.

In another embodiment, the system provided herein is used for the treatment or prophylaxis of one or more bacterial infections in a cystic fibrosis patient. In a further embodiment, the system provided herein comprises a liposomal aminoglycoside formulation. In a further embodiment, the aminoglycoside is amikacin.

In another embodiment, the system provided herein is used for the treatment or prophylaxis of one or more bacterial infections in a patient with bronchiectasis. In a further embodiment, the system provided herein comprises a liposomal aminoglycoside formulation. In a further embodiment, the aminoglycoside is amikacin or amikacin sulfate.

In yet another embodiment, the system provided herein is used for the treatment or prophylaxis of Pseudomonas aeruginosa lung infections in non-CF bronchiectasis patients. In a further embodiment, the system provided herein comprises a liposomal aminoglycoside formulation. In a further embodiment, the aminoglycoside is amikacin.

As provided herein, the present invention provides aminoglycoside formulations administered via inhalation. In one embodiment, the MMAD of the aerosol is about 3.2 μm to about 4.2 μm, as measured by the Anderson Cascade Impactor (ACI), or about 4.4 μm to about 4.9 μm, as measured by the Next Generation Impactor (NGI).

In one embodiment, the nebulization time of an effective amount of an aminoglycoside formulation provided herein is less than 20 minutes, less than 18 minutes, less than 16 minutes or less than 15 minutes. In one embodiment, the nebulization time of an effective amount of an aminoglycoside formulation provided herein is less than 15 minutes or less than 13 minutes. In one embodiment, the nebulization time of an effective amount of an aminoglycoside formulation provided herein is about 13 minutes.

In one embodiment, the formulation described herein is administered once daily to a patient in need thereof.

### EXAMPLES

The present invention is further illustrated by reference to the following Examples. However, it should be noted that these Examples, like the embodiments described above, are illustrative and are not to be construed as restricting the scope of the invention in any way.

### Example 1

### Comparison of Nebulizer Reservoir Volumes

In this example, the aerosol generator was an investigational eFlow® nebulizer, modified for use with liposomal aminoglycoside formulations provided herein, of Pari Pharma GmbH, Germany. A first aerosol generator had an initial volume of the liquid reservoir V_{RI} of 13 mL (A), a second one of 17 mL (B), a third one of 22 mL (C) and a fourth one of 20 mL (D). That is the increased volume V_{RN} of the first one had 15.5 mL, the second one 19.5 mL, the third 24.5 mL and the fourth 22.5 mL.

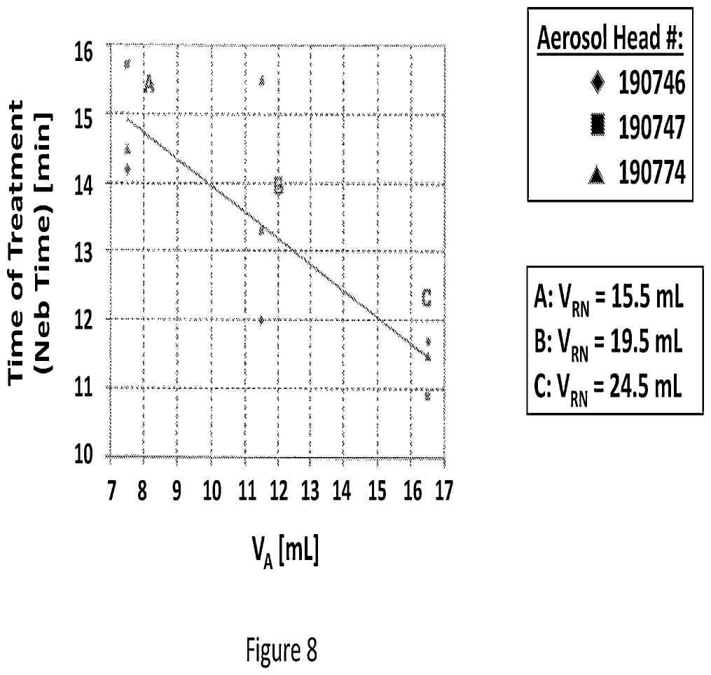

8 mL of a liposomal amikacin formulation was poured into the liquid reservoir 10. As shown in FIG. 8, an air cushion of 8 mL resulted in an aerosol generation time period upon complete emission of 8 mL of the formulation in the liquid reservoir of between 14 and 16 minutes. An air cushion of 12 mL, however, decreased the aerosol generation time to a range between 12 and approximately 13 minutes. The air cushion of 17 mL further decreases the aerosol generation time to an amount between 10 and 12 minutes (FIG. 6).

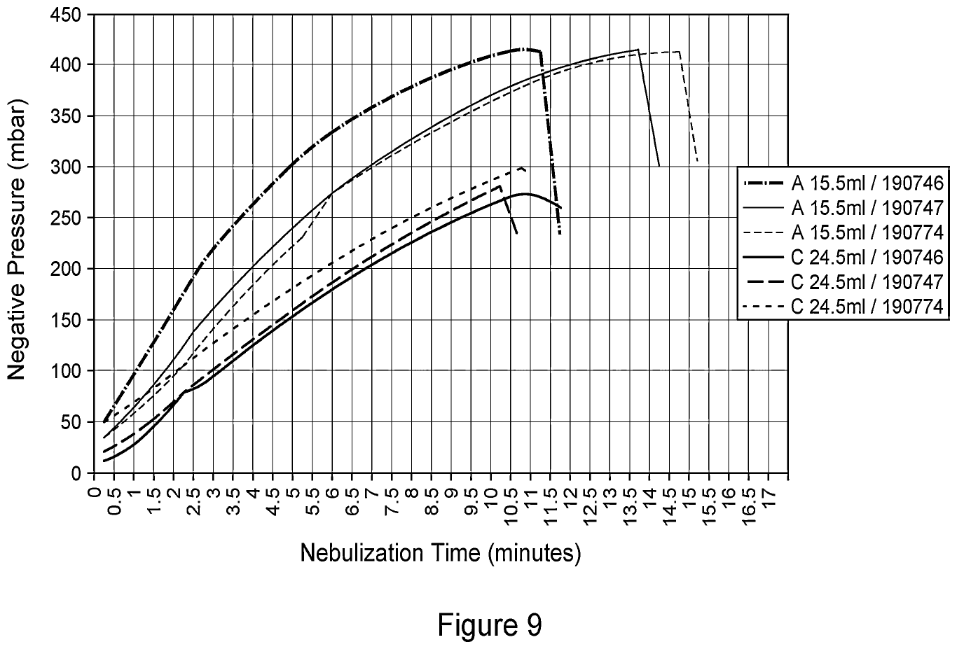

Further, the first (A) and third (C) version of the aerosol generator had been used together with 8 mL of the liposomal amikacin formulation. An initial negative pressure of equal to or less than 50 mbar was generated within the liquid reservoir. In addition, the negative pressure was measured during the aerosol generation and is shown over the aerosol generation time in FIG. 9. In other words, FIG. 9 shows experimental data comparing the negative pressure range during the aerosol generation time for a liquid reservoir (C) having a volume V_{RN} of 24.5 mL and a liquid reservoir (A) having a volume V_{RN} of 15.5 mL. The initial amount of amikacin formulation V_{L} was 8 mL and the initial negative pressure was about 50 mbar. The graph indicates that a larger air cushion prevents the negative pressure from increasing above a critical value of 300 mbar.

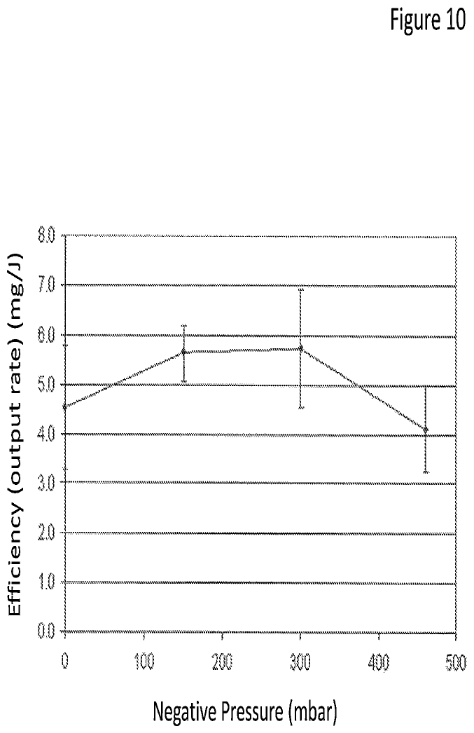

The dependence of aerosol generator efficiency (proportional to liquid output rate or total output rate) on different negative pressures was measured with the nebulizer described above. A liposomal amikacin formulation having a viscosity in the range of 5.5 to 14.5 mPa×s at sheer forces between 1.1 and 7.4 Pa (thixotrope) was used in the experiment. As shown in FIG. 10, the efficiency is optimum in a negative pressure range between 150 mbar and 300 mbar. As also shown in FIG. 10, the efficiency decreases at a negative pressure below approximately 150 mbar and at a negative pressure of above 300 mbar.

Furthermore, the same liposomal amikacin formulation as in FIG. 8 was used in four different aerosol generators based on the modified eFlow®, wherein the first aerosol generator (A) is a modified cFlow® with an increased volume V_{RN} of the liquid reservoir of 19.5 mL and filled with 8 mL of the liposomal amikacin formulation.

The second aerosol generator (B) had a reservoir with an increased volume V_{RN} of 16 mL filled with 8 mL of the mentioned liposomal amikacin formulation, the third aerosol generator (C) one had an increased volume V_{RN} of 24.5 mL, filled with 8 mL of the mentioned liquid. The fourth aerosol generator had an increased volume V_{RN} of the liquid reservoir of 22.5 mL, and was filled with 8 mL of the aforementioned liposomal amikacin formulation.

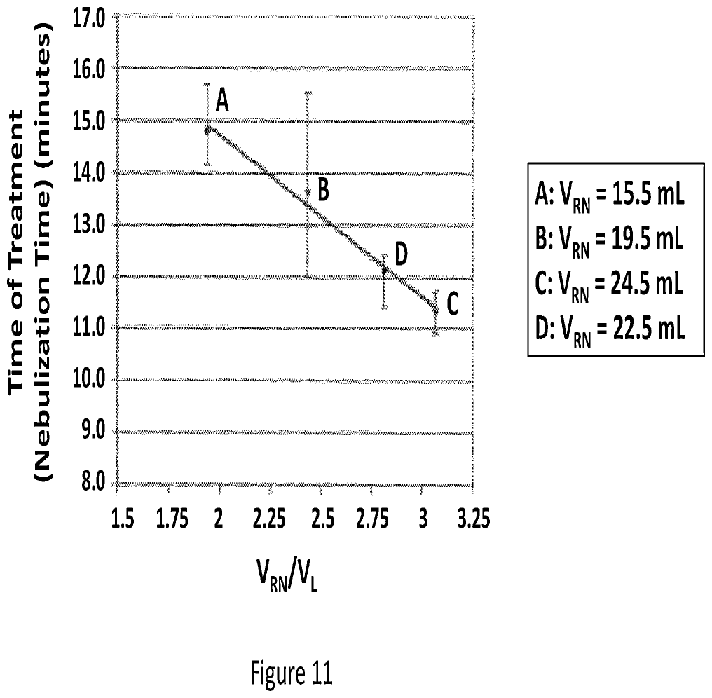

FIG. 11 shows experimental data of these four aerosol generators filled with 8 mL of the liposomal amikacin formulation. The results show the aerosol generation time for complete emission of the liposomal amikacin formulation within the liquid reservoir in relation to the ratio of the increased volume of the liquid reservoir (V_{RN}) to the initial volume of liquid in the liquid reservoir before use (V_{L}). FIG. 11 indicates that with the modified acrosol generator device (A) an aerosol generation time of approximately 16 minutes was required, whereas the aerosol generation time decreased with an increased ratio V_{RN}/V_{L}. The data also shows that the aerosol generation time could be reduced by approximately 4 minutes to below 12 minutes with the third aerosol generator device (C).

The data provided in Example 1 therefore indicates that a larger air cushion enables the operation of the aerosol generator for a longer time in an efficient negative pressure range so that the total aerosol generation time may significantly be reduced. Therefore, even large amounts of liquid such as 8 mL may be nebulized (emitted in form of aerosol) in a period of time below 12 minutes.

### Example 2

### Aerosol Properties of Amikacin Formulation

Eleven different lots of the liposomal amikacin formulation were examined with the modified cFlow® nebulizer (i.e., modified for use with the liposomal aminoglycoside formulations described herein) having a modified 40 mesh membrane fabricated as described herein, and a reservoir with an 8 mL liquid capacity and aforementioned air cushion. Cascade impaction was performed using either the ACI (Anderson Cascade Impactor) or the NGI (Next Generation Impactor) to establish aerosol properties: mass median acrodynamic diameter (MMAD), geometric standard deviation (GSD), and Fine-Particle-Fraction (FPF).

### Mass Median Acrodynamic Diameter (MMAD) Measurement With ACI

An Anderson Cascade Impactor (ACI) was used for MMAD measurements and the nebulization work was conducted inside a ClimateZone chamber (Westech Instruments Inc., GA) to maintain temperature and relative humanity % during nebulization. The ClimateZone was pre-set to a temperature of 18° C. and a relative humanity of 50%. The ACI was assembled and loaded inside the ClimateZone. A probe thermometer (VWR dual thermometer) was attached to the surface of ACI at stage 3 to monitor the temperature of ACI. Nebulization was started when the temperature of the ACI reached 18+0.5° C.

With the 8 mL handsets loaded with 8 mL, it was found that the ACI could not handle the whole 8 mL dose; i.e., amikacin liposomal formulation deposited on ACI plate 3 overflowed. It was determined that the percent drug distribution on each ACI stage was not affected by the amount of liposomal amikacin formulation collected inside the ACI as long as there was no liquid overflow at ACI stage 3 (data not shown). Therefore for nebulization, the nebulizer was either filled with 4 mL liposomal amikacin formulation and nebulized until empty or filled with 8mL of liposomal amikacin formulation and nebulized for about 6 minutes of collection time (i.e., ˜4 mL).

The nebulizate was collected at a flow rate of 28.3 L/min in the ACI which was cooled to 18° C. The nebulization time was recorded and the nebulization rate calculated based on the difference in weight (amount nebulized) divided by the time interval.

After the nebulizate was collected, ACI collection plates 0, 1, 2, 3, 4, 5, 6 and 7 were removed, and each was loaded into its own petri dish. An appropriate amount of extraction solution (20 mL for plates 2, 3, and 4, and 10 mL for plates 0, 1, 5, 6, and 7) was added to each Petri dish to dissolve the formulation deposited on each plate. Samples from plates 0, 1, 2, 3, 4, 5 and 6 were further diluted appropriately with mobile phase C for HPLC analysis. Sample from plate 7 was directly analyzed by HPLC without any further dilution. The ACI Filter was also transferred to a 20 mL vial and 10 mL extraction solution was added, and the capped vial vortexed to dissolve any formulation deposited on it. Liquid samples from the vial were filtered (0.2 μm) into HPLC vials for HPLC analysis. The induction port with connector was also rinsed with 10 mL extraction solution to dissolve the formulation deposited on it, and the sample was collected and analyzed by HPLC with 2 time dilution. Based on the amikacin amount deposited on each stage of the impactor, mass median aerodynamic diameter (MMAD), geometric standard deviation (GSD) and fine particle fraction (FPF) were calculated.

In the cases for nebulizers loaded with 8 mL and nebulized for 6 minutes fine particle dose (FPD) was normalized to the volume of formulation nebulized in order to compare FPD across all experiments. FPD (normalized to the volume of formulation nebulized) was calculated according to the following equation:

$$FPD ( normalized to volume nebulized ) ( \frac{mg}{mL} ) = \frac{Amikacin {Recovered}_{ACI} \times FPF ( mg )}{Arikace Nebulized ( g ) \div Density ( \frac{g}{mL} )}$$

### Mass Median Aerodynamic Diameter (MMAD) Measurement With NGI

A Next Generation Impactor (NGI) was also used for MMAD measurements and the nebulization work was conducted inside a ClimateZone chamber (Westech Instruments Inc., GA) to maintain temperature and RH % during nebulization. The ClimateZone was pre-set to a temperature of 18° C. and a relative humanity of 50%. The NGI was assembled and loaded inside the ClimateZone. A probe thermometer (VWR dual thermometer) was attached to the surface of NGI to monitor the temperature of NGI. Nebulization was started when the temperature of the NGI reached 18±0.5° C.

8 mL of the liposomal amikacin formulation was added to the nebulizer and nebulized. When there was no more aerosol observed, the timer was stopped. The nebulizate was collected at a flow rate of 15 L/min in the NGI which was cooled to 18° C. The nebulization time was recorded and the nebulization rate calculated based on the difference in weight (amount nebulized) divided by the time interval.

After aerosol collection was done, the NGI tray with tray holder was removed from NGI. An appropriate amount of extraction solution was added to NGI cups 1, 2, 3, 4, 5, 6, 7 and MOC to dissolve the formulation deposited on these cups. This material was transferred to a volumetric flask respectively. For NGI cups 1, 2, and 6, 25 ml volumetric flasks were used; for NIG cups 2, 3, 4, 50 ml volumetric flasks were used. More extraction solution was added to the cups and again transferred to the volumetric flask. This procedure was repeated several times in order to transfer formulation deposited on the NGI cup to the volumetric flask completely. The volumetric flasks were topped up to bring the final volume to either 25 ml or 50 ml and shaken well before sampled. Samples from cups 1, 2, 3, 4, 5, 6 and 7 were further diluted appropriately with mobile phase C for HPLC analysis. Sample from MOC was directly analyzed by HPLC without any further dilution. The NGI Filter was also transferred to a 20 mL vial and 10 mL extraction solution was added, and the capped vial vortexed to dissolve any formulation deposited on it. Liquid samples from the vial were filtered (0.2 micron) into HPLC vials for HPLC analysis. The Induction port with connector was also rinsed with 10 mL extraction solution to dissolve the formulation deposited on it, and the sample was collected and analyzed by HPLC with 11 time dilution.

Based on the amikacin amount deposited on each stage of the impactor, MMAD, GSD and FPF were calculated.

FPD was normalized to the volume of formulation nebulized in order to compare FPD across all experiments. FPD (normalized to the volume of formulation nebulized) was calculated according to the following equation:

$$FPD ( normalized to volume nebulized ) ( \frac{mg}{mL} ) = \frac{Amikacin {Recovered}_{ACI} \times FPF ( mg )}{Arikace Nebulized ( g ) \div Density ( \frac{g}{mL} )}$$

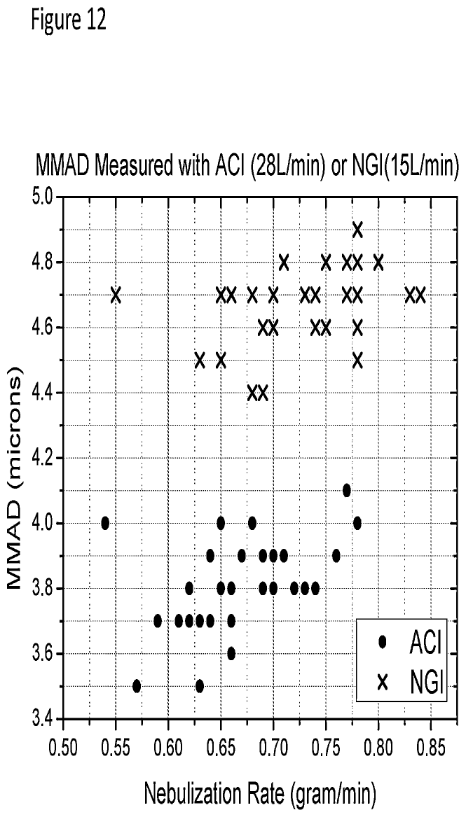

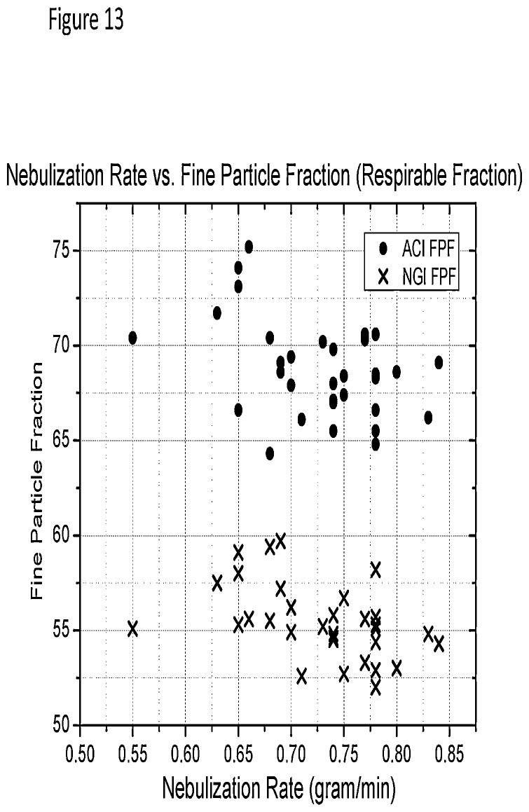

The results of these experiments are provided in FIGS. 12 and 13 and Table 5, below.

TABLE 5
Aerosol Characteristics

| | | ACI APSD Data | | | | | NGI APSD Data | | | | | Nebulization Data | | |
|---|---|---|---|---|---|---|---|---|---|---|---|---|---|---|
| | | Aerosol | Neb. | | | FPF | Aerosol | Neb. | | | FPF | Aerosol | Neb. | % Assoc. |
| Amikacin | | Head | Rate | MMAD | | <5 μm | Head | Rate | MMAD | | <5 μm | Head | Rate | Amikacin |
| Conc. | Run | ID | (g/min) | (μm) | GSD | (%) | ID | (g/min) | (μm) | GSD | (%) | ID | (g/min) | Post-Neb |
| 66.9 | 1 | J | 0.66 | 3.7 | 1.7 | 70.4 | J | 0.68 | 4.7 | 1.7 | 55.5 | A | 0.63 | 69.1 |
| mg/mL | 2 | K | 0.62 | 3.7 | 1.7 | 71.7 | K | 0.63 | 4.5 | 1.7 | 57.5 | B | 0.60 | 68.2 |
| | 3 | L | 0.65 | 4.0 | 1.8 | 66.1 | L | 0.71 | 4.8 | 1.7 | 52.6 | C | 0.56 | 69.9 |
| 70.8 | 1 | M | 0.64 | 3.9 | 1.7 | 67.1 | M | 0.74 | 4.7 | 1.7 | 54.8 | M | 0.65 | 64.5 |
| mg/mL | 2 | N | 0.68 | 4.0 | 1.8 | 64.8 | N | 0.78 | 4.9 | 1.7 | 52.0 | N | 0.67 | 66.3 |
| | 3 | O | 0.69 | 3.9 | 1.7 | 67.4 | O | 0.75 | 4.8 | 1.7 | 52.7 | O | 0.64 | 69.4 |
| 64.6 | 1 | C | 0.78 | 4.0 | 1.8 | 65.5 | A | 0.74 | 4.7 | 1.7 | 54.6 | G | 0.72 | 71.9 |
| mg/mL | 2 | D | 0.64 | 3.7 | 1.7 | 70.2 | B | 0.73 | 4.7 | 1.7 | 55.2 | H | 0.64 | 71.5 |
| | 3 | H | 0.62 | 3.7 | 1.7 | 70.6 | C | 0.78 | 4.7 | 1.7 | 54.4 | J | 0.68 | 71.8 |
| 68.5 | 1 | E | 0.69 | 3.8 | 1.7 | 69.4 | E | 0.70 | 4.6 | 1.7 | 56.2 | E | 0.60 | 69.1 |
| mg/mL | 2 | F | 0.78 | 4.0 | 1.8 | 66.2 | F | 0.83 | 4.7 | 1.7 | 54.8 | F | 0.67 | 70.4 |
| | 3 | G | 0.65 | 3.8 | 1.7 | 69.1 | G | 0.69 | 4.6 | 1.7 | 57.2 | G | 0.61 | 69.5 |
| 65.7 | 1 | V | 0.74 | 3.8 | 1.7 | 69.1 | V | 0.84 | 4.7 | 1.7 | 54.3 | M | 0.64 | 69.2 |
| mg/mL | 2 | W | 0.72 | 3.8 | 1.7 | 68.3 | W | 0.78 | 4.7 | 1.7 | 55.1 | N | 0.74 | 67.9 |
| | 3 | X | 0.70 | 3.9 | 1.7 | 68.0 | X | 0.74 | 4.7 | 1.7 | 54.5 | O | 0.63 | 68.6 |
| 66.8 | 1 | J | 0.63 | 3.7 | 1.8 | 70.6 | A | 0.77 | 4.8 | 1.7 | 53.3 | A | 0.70 | 73.2 |
| mg/mL | 2 | K | 0.59 | 3.7 | 1.8 | 70.4 | D | 0.55 | 4.7 | 1.7 | 55.1 | B | 0.70 | 72.4 |
| | 3 | L | 0.64 | 3.9 | 1.8 | 66.6 | H | 0.65 | 4.7 | 1.7 | 55.3 | C | 0.83 | 72.8 |
| 69.2 | 1 | S | 0.66 | 3.8 | 1.7 | 68.6 | U | 0.80 | 4.8 | 1.7 | 53.0 | S | 0.69 | 70.7 |
| mg/mL | 2 | T | 0.73 | 3.8 | 1.7 | 68.3 | V | 0.78 | 4.5 | 1.7 | 58.2 | T | 0.75 | 71.0 |
| | 3 | U | 0.54 | 4.0 | 1.8 | 65.5 | W | 0.78 | 4.7 | 1.7 | 55.3 | U | 0.80 | 71.1 |
| 71.4 | 1 | Q | 0.66 | 3.8 | 1.7 | 68.4 | M | 0.75 | 4.6 | 1.7 | 56.7 | P | 0.71 | 72.4 |
| mg/mL | 2 | R | 0.71 | 3.9 | 1.8 | 66.6 | N | 0.78 | 4.8 | 1.7 | 52.9 | Q | 0.68 | 70.0 |
| | 3 | S | 0.66 | 3.8 | 1.7 | 68.5 | O | 0.78 | 4.6 | 1.7 | 55.7 | R | 0.74 | 71.7 |
| 69.9 | 1 | C | 0.77 | 4.1 | 1.8 | 64.3 | J | 0.68 | 4.4 | 1.7 | 59.4 | A | 0.68 | 73.8 |
| mg/mL | 2 | D | 0.62 | 3.8 | 1.7 | 68.6 | K | 0.69 | 4.4 | 1.7 | 59.7 | B | 0.63 | 73.6 |
| | 3 | H | 0.61 | 3.7 | 1.7 | 70.3 | L | 0.77 | 4.7 | 1.7 | 55.6 | C | 0.70 | 75.7 |
| 72.2 | 1 | T | 0.70 | 3.8 | 1.7 | 69.8 | T | 0.74 | 4.6 | 1.7 | 55.8 | M | 0.65 | 67.9 |
| mg/mL | 2 | U | 0.76 | 3.9 | 1.7 | 67.0 | U | 0.74 | 4.7 | 1.7 | 54.8 | N | 0.71 | 70.3 |
| | 3 | X | 0.67 | 3.9 | 1.7 | 67.9 | X | 0.70 | 4.7 | 1.7 | 54.9 | P | 0.57 | 71.8 |
| 70.4 | 1 | C | 0.66 | 3.6 | 1.7 | 73.1 | J | 0.65 | 4.5 | 1.7 | 58.0 | H | 0.59 | 60.1 |
| mg/mL | 2 | D | 0.57 | 3.5 | 1.7 | 74.1 | K | 0.65 | 4.5 | 1.7 | 59.1 | J | 0.69 | 59.3 |
| | 3 | E | 0.63 | 3.5 | 1.7 | 75.2 | L | 0.66 | 4.7 | 1.7 | 55.6 | K | 0.63 | 58.5 |

### Example 3

### Nebulization Rate Study

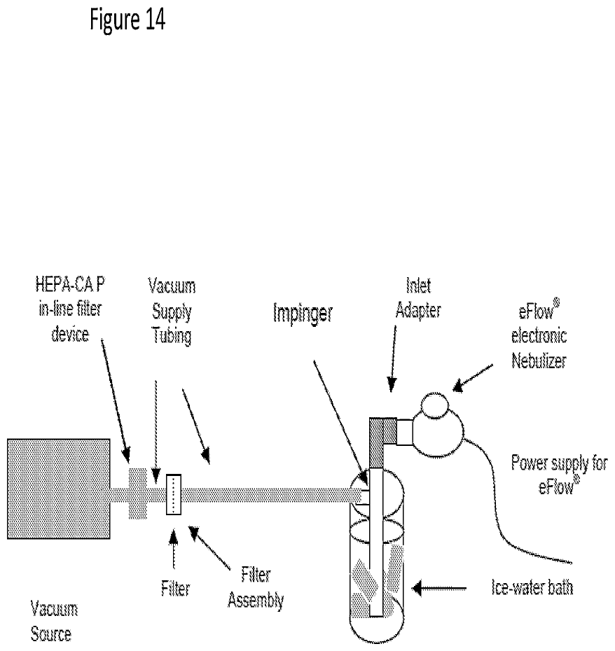

Nebulization rate studies (grams of formulation nebulized per minute) were conducted in a biosafety cabinet (Model 1168, Type B2, FORMA Scientific). The assembled nebulizer (handset with mouth piece and aerosol head) was first weighed empty (W₁), then a certain volume of formulation was added and the nebulizer device was weighed again (W₂). The nebulizer and timer were started and the formulations nebulized were collected in a chilled impinger at a flow rate of ˜8 L/min (see FIG. 14 for details of experimental setup). When there was no more aerosol observed, the timer was stopped. The nebulizer was weighed again (W₃), and the time of nebulization (t) was recorded. Total formulation nebulized was calculated as W₂−W₃ and total drug residue after nebulization was calculated as W₃−W₁. The nebulization rate of formulation was calculated according to the following equation:

$$Nebulization Rate ( \frac{g}{min} ) = \frac{{W}_{2} - {W}_{3}}{t}$$

Nebulization rates in g/min., as well as other related results, for liposomal amikacin nebulized using a nebulizer fabricated according to the specification (twenty four aerosol heads were selected and were used in these studies) are captured in Table 6.

TABLE 6
Formulation nebulization rates (g/min)

| | | Neb | Formulation | Neb |
|---|---|---|---|---|
| | Aerosol | Time | Nebulized | Rate |
| Run | Head # | (min) | (g) | (g/min) |
| 1 | 1 | 11.90 | 7.7346 | 0.65 |
| 2 | 2 | 11.58 | 8.0573 | 0.70 |
| 3 | 3 | 10.87 | 8.0029 | 0.74 |
| 4 | 4 | 13.63 | 7.9359 | 0.58 |
| 5 | 5 | 12.60 | 8.0577 | 0.64 |
| 6 | 6 | 12.62 | 8.0471 | 0.64 |
| 7 | 7 | 14.23 | 8.073 | 0.57 |
| 8 | 8 | 14.67 | 8.0872 | 0.55 |
| 9 | 9 | 13.58 | 7.9235 | 0.58 |
| 10 | 10 | 12.28 | 7.9649 | 0.65 |
| 11 | 11 | 12.33 | 8.1872 | 0.66 |
| 12 | 12 | 13.17 | 8.1694 | 0.62 |
| 13 | 1 | 11.22 | 7.9991 | 0.71 |
| 14 | 2 | 11.90 | 8.1392 | 0.68 |
| 15 | 3 | 12.17 | 8.0162 | 0.66 |
| 16 | 4 | 12.90 | 8.0174 | 0.62 |
| 17 | 5 | 11.22 | 7.893 | 0.70 |
| 18 | 6 | 10.23 | 8.0401 | 0.79 |
| 19 | 7 | 12.55 | 8.0988 | 0.65 |
| 20 | 8 | 14.88 | 7.8781 | 0.53 |
| 21 | 9 | 13.68 | 8.1678 | 0.60 |
| 22 | 10 | 12.33 | 8.2253 | 0.67 |
| 23 | 11 | 12.60 | 8.0783 | 0.64 |
| 24 | 12 | 11.83 | 7.946 | 0.67 |
| 25 | 1 | 11.92 | 8.1703 | 0.69 |
| 26 | 2 | 11.95 | 7.9837 | 0.67 |
| 27 | 3 | 13.63 | 8.1536 | 0.60 |
| 28 | 4 | 11.90 | 7.9376 | 0.67 |
| 29 | 5 | 12.27 | 8.1727 | 0.67 |
| 30 | 6 | 12.27 | 8.0875 | 0.66 |
| 31 | 7 | 13.65 | 8.0767 | 0.59 |
| 32 | 8 | 15.80 | 8.1183 | 0.51 |
| 33 | 9 | 13.65 | 8.1373 | 0.60 |
| 34 | 10 | 12.98 | 7.8864 | 0.61 |
| 35 | 11 | 11.63 | 8.1445 | 0.70 |
| 36 | 12 | 12.95 | 8.0232 | 0.62 |
| 37 | 13 | 12.80 | 7.9098 | 0.62 |
| 38 | 14 | 10.25 | 8.0328 | 0.78 |
| 39 | 15 | 12.13 | 7.9911 | 0.66 |
| 40 | 16 | 12.33 | 8.1756 | 0.66 |
| 41 | 17 | 12.47 | 7.9417 | 0.64 |
| 42 | 18 | 13.17 | 7.9046 | 0.60 |
| 43 | 19 | 13.92 | 7.5367 | 0.54 |
| 44 | 20 | 11.47 | 8.1466 | 0.71 |
| 45 | 21 | 11.67 | 7.9366 | 0.68 |
| 46 | 22 | 13.17 | 8.0613 | 0.61 |
| 47 | 23 | 12.77 | 7.8596 | 0.62 |
| 48 | 24 | 12.25 | 8.0552 | 0.66 |
| 49 | 13 | 13.67 | 7.9379 | 0.58 |
| 50 | 14 | 10.55 | 8.0221 | 0.76 |
| 51 | 15 | 11.80 | 8.0555 | 0.68 |
| 52 | 16 | 10.08 | 8.1639 | 0.81 |
| 53 | 17 | 11.08 | 7.9121 | 0.71 |
| 54 | 18 | 12.28 | 8.017 | 0.65 |
| 55 | 19 | 11.40 | 7.9415 | 0.70 |
| 56 | 20 | 12.17 | 8.211 | 0.67 |
| 57 | 21 | 11.45 | 8.18 | 0.71 |
| 58 | 22 | 12.03 | 7.8946 | 0.66 |
| 59 | 23 | 12.83 | 8.0771 | 0.63 |
| 60 | 24 | 11.97 | 7.9936 | 0.67 |
| 61 | 13 | 12.38 | 8.0054 | 0.65 |
| 62 | 14 | 10.53 | 8.0492 | 0.76 |
| 63 | 15 | 11.82 | 7.8161 | 0.66 |
| 64 | 16 | 11.83 | 8.1169 | 0.69 |
| 65 | 17 | 12.67 | 8.1778 | 0.65 |
| 66 | 18 | 12.03 | 8.2436 | 0.69 |
| 67 | 19 | 13.17 | 7.8821 | 0.60 |
| 68 | 20 | 12.17 | 8.2397 | 0.68 |
| 69 | 21 | 11.78 | 8.1814 | 0.69 |
| 70 | 22 | 11.78 | 8.3443 | 0.71 |
| 71 | 23 | 13.17 | 8.1699 | 0.62 |
| 72 | 24 | 11.50 | 8.0413 | 0.70 |
| Average | | 12.4 ± 1.1 | 8.0 ± 0.1 | 0.66 ± 0.06 |

### Example 4

### Percent of Associate Amikacin Post-Nebulization and Nebulizate Characterization

The free and liposomal complexed amikacin in the nebulizate of Example 3 was measured. As mentioned in Example 3, the nebulizate was collected in a chilled impinger at a flow rate of 8 L/min (FIG. 14).

The nebulizate collected in the impinger was rinsed with 1.5% NaCl and transferred to a 100 mL or 50-mL volumetric flask. The impinger was then rinsed several times with 1.5% NaCl in order to transfer all the formulation deposited in the impinger to the flask. To measure the free amikacin concentration of the nebulizate, 0.5 mL of the diluted nebulizate inside the volumetric flask was taken and loaded to an Amicon® Ultra-0.5 mL 30K centrifugal filter device (regenerated cellulose, 30K MWCO, Millipore) and this device was centrifuged at 5000 G at 15° C. for 15 minutes. An appropriate amount of filtrate was taken and was diluted 51 times with mobile phase C solution. Amikacin concentration was determined by HPLC. To measure total amikacin concentration of the nebulizate, an appropriate amount of the diluted nebulizate inside the volumetric flask was taken and diluted (also dissolved) 101 times in extraction solution (perfluoropentanoic acid: 1-propanol: water (25:225:250, v/v/v)) and the amikacin concentration determined by HPLC.

The percent associated amikacin post-nebulization was calculated by the following equation:

$$\% Associated = \frac{{Concentration}_{Total} - {Concentration}_{Free}}{{Concentration}_{Total}} \times 100$$

The percent associated amikacin post-nebulization and total dose recovery from nebulization experiments described in Table 6 are summarized in Table 7. Corresponding nebulization rates were also included in Table 7.

TABLE 7
Percent associated amikacin post-nebulization
and total dose recovered

| | Aerosol | % | Recovered | Neb Rate |
|---|---|---|---|---|
| Run | Head # | Associated | % | (g/min) |
| 1 | 1 | 65.7 | 104 | 0.65 |
| 2 | 2 | 65.1 | 97 | 0.70 |
| 3 | 3 | 64.5 | 96 | 0.74 |
| 4 | 4 | 66.1 | 97 | 0.58 |
| 5 | 5 | 62.1 | 92 | 0.64 |
| 6 | 6 | 65.5 | 95 | 0.64 |
| 7 | 7 | 63.5 | 94 | 0.57 |
| 8 | 8 | 60.4 | 92 | 0.55 |
| 9 | 9 | 65.0 | 93 | 0.58 |
| 10 | 10 | 72.7 | 102 | 0.65 |
| 11 | 11 | 64.9 | 92 | 0.66 |
| 12 | 12 | 66.7 | 97 | 0.62 |
| 13 | 1 | 67.1 | 102 | 0.71 |
| 14 | 2 | 64.2 | 97 | 0.68 |
| 15 | 3 | 68.8 | 98 | 0.66 |
| 16 | 4 | 65.5 | 94 | 0.62 |
| 17 | 5 | 66.1 | 98 | 0.70 |
| 18 | 6 | 65.7 | 94 | 0.79 |
| 19 | 7 | 65.5 | 100 | 0.65 |
| 20 | 8 | 64.8 | 95 | 0.53 |
| 21 | 9 | 60.3 | 94 | 0.60 |
| 22 | 10 | 59.1 | 95 | 0.67 |
| 23 | 11 | 63.3 | 95 | 0.64 |
| 24 | 12 | 66.3 | 98 | 0.67 |
| 25 | 1 | 66.4 | 104 | 0.69 |
| 26 | 2 | 63.5 | 93 | 0.67 |
| 27 | 3 | 62.9 | 93 | 0.60 |
| 28 | 4 | 64.2 | 93 | 0.67 |
| 29 | 5 | 64.9 | 99 | 0.67 |
| 30 | 6 | 68.2 | 98 | 0.66 |
| 31 | 7 | 61.0 | 96 | 0.59 |
| 32 | 8 | 59.9 | 96 | 0.51 |
| 33 | 9 | 63.0 | 95 | 0.60 |
| 34 | 10 | 58.1 | 95 | 0.61 |
| 35 | 11 | 66.1 | 98 | 0.70 |
| 36 | 12 | 64.2 | 98 | 0.62 |
| 37 | 13 | 65.6 | 100 | 0.62 |
| 38 | 14 | 68.9 | 96 | 0.78 |
| 39 | 15 | 63.7 | 97 | 0.66 |
| 40 | 16 | 64.7 | 97 | 0.66 |
| 41 | 17 | 69.1 | 97 | 0.64 |
| 42 | 18 | 70.2 | 94 | 0.60 |
| 43 | 19 | 61.2 | 93 | 0.54 |
| 44 | 20 | 63.4 | 91 | 0.71 |
| 45 | 21 | 67.7 | 99 | 0.68 |
| 46 | 22 | 66.7 | 96 | 0.61 |
| 47 | 23 | 67.2 | 93 | 0.62 |
| 48 | 24 | 69.6 | 98 | 0.66 |
| 49 | 13 | 66.2 | 102 | 0.58 |
| 50 | 14 | 66.9 | 97 | 0.76 |
| 51 | 15 | 66.7 | 96 | 0.68 |
| 52 | 16 | 64.7 | 96 | 0.81 |
| 53 | 17 | 65.1 | 96 | 0.71 |
| 54 | 18 | 67.6 | 98 | 0.65 |
| 55 | 19 | 66.7 | 97 | 0.70 |
| 56 | 20 | 63.6 | 99 | 0.67 |
| 57 | 21 | 68.1 | 101 | 0.71 |
| 58 | 22 | 64.8 | 99 | 0.66 |
| 59 | 23 | 66.2 | 97 | 0.63 |
| 60 | 24 | 67.4 | 103 | 0.67 |
| 61 | 13 | 64.2 | 99 | 0.65 |
| 62 | 14 | 68.7 | 101 | 0.76 |
| 63 | 15 | 66.0 | 100 | 0.66 |
| 64 | 16 | 67.7 | 103 | 0.69 |
| 65 | 17 | 66.4 | 100 | 0.65 |
| 66 | 18 | 66.2 | 98 | 0.69 |
| 67 | 19 | 68.3 | 100 | 0.60 |
| 68 | 20 | 67.9 | 101 | 0.68 |
| 69 | 21 | 67.1 | 98 | 0.69 |
| 70 | 22 | 66.2 | 101 | 0.71 |
| 71 | 23 | 67.0 | 97 | 0.62 |
| 72 | 24 | 68.0 | 100 | 0.70 |
| Average | | 65.5 ± 2.6 | 97 ± 3 | 0.66 ± 0.06 |

The total concentration of amikacin in the liposomal amikacin formulation was measured during this study with the rest of the samples using the same HPLC and amikacin standards. The value obtained was 64 mg/mL amikacin. The % associated amikacin post-nebulization values ranged from 58.1% to 72.7%, with an average value of 65.5±2.6%; for 8 mL liposomal amikacin formulation nebulized, the total recovered amount of amikacin ranged from 426 mg to 519 mg, with an average value of 476±17 mg; the calculated amount of amikacin nebulized (according to the weight of the liposomal amikacin formulation nebulized in Table 7) ranged from 471 mg to 501 mg, with an average value of 490±8 mg; the total amikacin recovery ranged from 91% to 104%, with an average value of 97±3% (n=72).

### Liposome Size

The liposomal amikacin formulation (64 mg/mL amikacin), either pre-nebulized or post-nebulized, was diluted appropriately with 1.5% NaCl and the liposome particle size was measured by light scattering using a Nicomp 380 Submicron Particle Sizer (Nicomp, Santa Barbara, CA).

The liposome sizes post-nebulization of the liposomal amikacin formulation aerosolized with twenty four nebulizer aerosol heads with 8 mL reservoir handsets were measured. The liposome size ranged from 248.9 nm to 288.6 nm, with an average of 264.8±6.7 nm (n=72). These results are provided in Table 8. The pre-nebulization liposome mean diameter was approximately 285 nm (284.5 nm±6.3 nm).

TABLE 8
Liposome size post-nebulization

| | Aerosol Head | Mean Diameter |
|---|---|---|
| Run | # | (nm) |
| 1 | 1 | 270.9 |
| 2 | 2 | 274.6 |
| 3 | 3 | 253.9 |
| 4 | 4 | 256.3 |
| 5 | 5 | 274.0 |
| 6 | 6 | 273.6 |
| 7 | 7 | 260.0 |
| 8 | 8 | 268.1 |
| 9 | 9 | 264.7 |
| 10 | 10 | 254.8 |
| 11 | 11 | 266.9 |
| 12 | 12 | 270.0 |
| 13 | 1 | 269.6 |
| 14 | 2 | 271.2 |
| 15 | 3 | 254.6 |
| 16 | 4 | 270.7 |
| 17 | 5 | 260.8 |
| 18 | 6 | 252.3 |
| 19 | 7 | 267.8 |
| 20 | 8 | 265.0 |
| 21 | 9 | 261.5 |
| 22 | 10 | 258.0 |
| 23 | 11 | 248.9 |
| 24 | 12 | 262.4 |
| 25 | 1 | 266.0 |
| 26 | 2 | 270.4 |
| 27 | 3 | 268.6 |
| 28 | 4 | 266.6 |
| 29 | 5 | 259.4 |
| 30 | 6 | 265.2 |
| 31 | 7 | 262.4 |
| 32 | 8 | 257.7 |
| 33 | 9 | 264.1 |
| 34 | 10 | 258.5 |
| 35 | 11 | 273.4 |
| 36 | 12 | 260.2 |
| 37 | 13 | 266.0 |
| 38 | 14 | 270.2 |
| 39 | 15 | 268.2 |
| 40 | 16 | 266.2 |
| 41 | 17 | 265.5 |
| 42 | 18 | 268.5 |
| 43 | 19 | 263.3 |
| 44 | 20 | 257.8 |
| 45 | 21 | 271.3 |
| 46 | 22 | 266.2 |
| 47 | 23 | 270.6 |
| 48 | 24 | 269.7 |
| 49 | 13 | 269.1 |
| 50 | 14 | 265.7 |
| 51 | 15 | 258.7 |
| 52 | 16 | 268.0 |
| 53 | 17 | 266.2 |
| 54 | 18 | 254.0 |
| 55 | 19 | 263.9 |
| 56 | 20 | 265.3 |
| 57 | 21 | 264.5 |
| 58 | 22 | 266.5 |
| 59 | 23 | 264.8 |
| 60 | 24 | 271.7 |
| 61 | 13 | 259.8 |
| 62 | 14 | 268.8 |
| 63 | 15 | 265.9 |
| 64 | 16 | 274.7 |
| 65 | 17 | 256.2 |
| 66 | 18 | 269.7 |
| 67 | 19 | 257.7 |
| 68 | 20 | 255.7 |
| 69 | 21 | 264.8 |
| 70 | 22 | 288.6 |
| 71 | 23 | 252.1 |
| 72 | 24 | 263.4 |
| Average | | 264.8 ± 6.7 |

All, documents, patents, patent applications, publications, product descriptions. and protocols which are cited throughout this application are incorporated herein by reference in their entireties for all purposes.

The embodiments illustrated and discussed in this specification are intended only to teach those skilled in the art the best way known to the inventors to make and use the invention. Modifications and variation of the above-described embodiments of the invention are possible without departing from the invention, as appreciated by those skilled in the art in light of the above teachings. It is therefore understood that, within the scope of the claims and their equivalents, the invention may be practiced otherwise than as specifically described.

1-29. (canceled)

30. A method for treating or prophylaxis against a pulmonary infection in a patient, the method comprising:
aerosolizing a pharmaceutical formulation comprising a liposomal complexed aminoglycoside, wherein the pharmaceutical formulation is an aqueous dispersion and is aerosolized at a rate greater than about 0.53 gram per minute, and
administering the aerosolized pharmaceutical formulation to the lungs of the patient;
wherein the aerosolized pharmaceutical formulation comprises a mixture of free aminoglycoside and liposomal complexed aminoglycoside, and
the lipid component of the liposome consists of electrically neutral lipids, and
the MMAD of the aerosol is less than about 4.2 μm, as measured by the ACI, or less than about 4.9 μm, as measured by the NGI.

31-70. (canceled)

71. A liposomal aminoglycoside aerosol comprising an aminoglycoside and a liposome comprising DPPC and cholesterol, wherein about 65% to about 75% of the aminoglycoside is liposomal complexed and the liposomal aminoglycoside aerosol is generated at a rate greater than about 0.53 gram per minute.

72-146. (canceled)
